(19) United States
(12) Patent Application Publication
(10) Pub. No.: US 2023/0174961 A1
(43) Pub. Date: Jun. 8, 2023

(54) TRANSCRIPTIONAL ROADBLOCKS FOR GENE EDITING AND METHODS OF USING THE SAME

(71) Applicant: Inscripta, Inc., Boulder, CO (US)

(72) Inventors: Andrew Garst, Boulder, CO (US); Christian Siltanen, Boulder, CO (US)

(73) Assignee: Inscripta, Inc., Boulder, CO (US)

(21) Appl. No.: 18/101,543

(22) Filed: Jan. 25, 2023

Related U.S. Application Data
(63) Continuation of application No. 17/521,739, filed on Nov. 8, 2021, now abandoned.
(60) Provisional application No. 63/112,066, filed on Nov. 10, 2020.

(51) Int. Cl. C12N 9/22; C12N 15/90; C12N 15/11

(52) U.S. Cl. CPC C12N 9/22; C12N 15/11; C12N 15/902; C12N 2310/20

(57) ABSTRACT

The present disclosure relates to automated multi-module instruments, compositions and methods for performing nucleic acid-guided nuclease editing; specifically, the disclosure provides nucleic acid cassettes, plasmids, vectors, and compositions comprising the same that employ homologous recombination for genome engineering by having a CRISPR nuclease cause a specific DSB while tethered to a repair nucleic acid.

### RELATED CASES

This application is a continuation application of U.S. Pat. Application No. 17/521,739, filed Nov. 8, 2021, which claims priority to USSN 63/112,066, filed 10 Nov. 2020, entitled “TRANSCRIPTIONAL ROADBLOCKS FOR GENE EDITING AND METHODS OF USING THE SAME”, which are incorporated herein in their entireties.

### FIELD OF THE INVENTION

The present disclosure relates to automated multi-module instruments, compositions and methods for performing nucleic acid-guided nuclease editing; specifically, the disclosure relates to gene editing cassettes that facilitate the presence of a repair DNA template near the site of cleavage of a CRISPR nuclease.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

The ability to make precise, targeted changes to the genome of living cells has been a long-standing goal in biomedical research and development. Recently, various nucleases have been identified that allow for manipulation of gene sequences; and hence gene function. The nucleases include nucleic acid-guided nucleases (i.e., CRISPR nucleases), which enable researchers to generate permanent edits in live cells. Editing efficiencies frequently correlate with the level of expression of guide RNAs (gRNAs) in the cell. That is, the higher the expression level of gRNA, the better the editing efficiency. Moreover, editing efficiencies also correlate with the gRNAs being localized in the nucleus; that is, for efficient editing to occur, the gRNAs must remain in the nucleus to direct editing, rather than being exported from the nucleus to the cytoplasm.

There is thus a need in the art of nucleic acid-guided nuclease gene editing for improved methods, compositions, modules and instruments for targeting components of gene editing systems, e.g., repair templates, gRNAs, and nucleic acid guided nucleases, to a nucleus of a cell and keeping the repair system assembled. The present invention satisfies this need.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

The present disclosure provides gene editing vectors, compositions, automated methods and multi-module automated instrumentation for performing gene editing, including gene editing in recursive editing protocols. A significant hurdle in obtaining high efficiencies of site-specific mutagenesis with CRISPR systems is the low efficiency of homology-directed recombination, which is limited by low concentration of repair DNA template at the cleavage site. The present invention solves this challenge by providing compositions that have a site-specific nuclease tethered to a repair DNA template, thus providing a repair template sequence at a sufficient distance from the cleavage site for efficient site mutagenesis. The tethering contemplated by the disclosure generally occurs by engineering a transcriptional road-block on a repair nucleic acid template, typically a double-stranded DNA sequence (dsDNA), and transcribing a gRNA sequence and another sequence from the repair nucleic acid template up until the RNA polymerase- (RNAP-) driving transcription becomes stalled at the transcriptional roadblock. The process generates a gRNA functionally tethered at a position in the template sufficiently close to a cleavage site by a nuclease.

This is significant because precise gene editing often relies on the ability of a system to guide and control the HDR process within a cell. After a CRISPR complex generates a double-strand break (DSB), many organisms can use endogenous DNA repair mechanisms to join the broken ends together. There are two main pathways that the cells follow to repair the break: non-homologous end joining (NHEJ) and homology-directed repair (HDR). As its name implies, the NHEJ pathway joins DSB ends employing a homologous template. However, NHEJ activity is error prone and introduces small insertions or deletions (indels) at the break point, potentially disrupting a target gene’s normal function. NHEJ is therefore generally most useful for creating gene knockouts. Introducing precise, sequence-specific edits or changes require HDR, the second repair pathway. HDR requires a repair DNA fragment containing sequences often identical, or highly homologous, to those flanking the break point. These sequences, often referred to as homology arms, enable homologous recombination by the endogenous cellular machinery. Consequently, HDR can repair DSBs in an error-free manner. The repair DNA template can carry a change in sequence between the homology arms, or it can carry much larger fragments for insertion at or near the break point. In sum, systems that can effectively guide the HDR process are required for precise gene editing.

Thus in some embodiments, there is provided a composition for homologous recombination based editing of a cell comprising: a double-stranded repair nucleic acid (dsDNA) cassette having: a sequence encoding a guide RNA for recruiting an endonuclease; a sequence homologous to a target region of a target cell, wherein the sequence homologous to the target region has at least one nucleic acid base variation compared to the target region of the target cell; one or more transcriptional roadblock moieties at a transcriptional roadblock within a distance of a putative double-strand cleavage site for the endonuclease. In such cases, transcription of the cassette within a cell provides for real-time generation of the transcriptional roadblock within a live cell. See FIG. 1C.

In other cases, the transcriptional roadblock is “pre-assembled.” In such instances, the disclosure provides a composition for homologous recombination-based editing of a live cell comprising: a dsDNA repair nucleic acid cassette having a sequence encoding a guide RNA for recruiting an endonuclease; and a sequence homologous to a target region of a target cell, wherein the sequence homologous to the target region has at least one nucleic acid base variation compared to the target region of the target cell; whereby the dsDNA repair nucleic acid is tethered by an RNA polymerase (RNAP) molecule stalled at a transcriptional roadblock to a nuclease via binding of the nuclease to RNA transcribed from the dsDNA repair nucleic acid. In such instances, the dsDNA repair nucleic acid sequence is tethered to the transcribed gRNA sequence by the RNAP with a 1:1 stoichiometry.

Yet, in other cases, the disclosure contemplates the creation of a single-stranded repair template (ssDNA), of the sense and nonsense polarity, by synthetically generating a ssDNA template having a gRNA and a sequence homologous to a target region up until a transcriptional roadblock.

In preferred embodiments, the transcriptional roadblock is a non-covalent interaction between a transcriptional roadblock ligand and a transcriptional roadblock ligand-binding moiety. Such non-covalent interactions between the transcriptional roadblock ligand and the transcriptional roadblock ligand binding moiety can provide for strong binding of the ligand-binding moiety to the roadblock ligand, for instances binding having dissociation constants (K_{d}) on the order of 10⁻¹² mol/L, on the order of 10⁻¹³ mol/L, or on the order of 10⁻¹⁴ mol/L. In specific instances, the transcriptional roadblock ligand is a biotin molecule and the ligand-binding moiety is a streptavidin molecule or an avidin molecule; however, the disclosure also contemplates other suitable transcriptional roadblocks for stalling the transcription of an RNA polymerase at a pre-determined location on a template, including a non-canonical nucleobase or a stretch of non-canonical nucleobases.

The disclosure contemplates designing the transcriptional roadblock within a suitable distance to a cleavage site for a CRISPR endonuclease, and the suitable distance can be within 500 bases, within 250 bases within 100 bases, or within 50 bases of the transcriptional roadblock. In most embodiments, the sequence homologous to the target region is between 50 base pairs to 500 base pairs long. Further the sequence homologous to the target region has between one to five variations, between one to ten variations, between one to fifteen variations, between one to twenty variations, between one to twenty-five variations, between one to thirty variations, between one to thirty-five variations, between one to forty variations, between one to forty-five variations, between one to fifty variations, between one to fifty-five variations, or between one to sixty variations compared to the target cell. The variation can be a deletion of a nucleobase, an addition of a nucleobase, or a replacement of a nucleobase compared to the target region of the target cell. The variation can also be at least one nucleic acid base variation compared to the target region of the target cell is designed to introduce a silent mutation on the target cell. In preferred embodiments, the dsDNA cassette further comprises a sequence encoding the nuclease. In such cases, the silent mutation may provide a site conferring immunity to further editing by the nuclease, such as a change in a PAM sequence for the nuclease. In preferred embodiments, the endonuclease is selected from the group consisting of MAD7, Cas9, or Cas12.

In addition, the compositions of the disclose are suitable for use with automated multi-module instruments for performing multiplex nucleic acid-guided nuclease editing, which encompasses compositions comprising a plurality of double-stranded repair nucleic acid (dsDNA) molecules for multiplex gene editing. The plurality of double-stranded repair nucleic acid (dsDNA) molecules in the composition can target at least 2, at least 10, at least 50, or at least 100 distinct target regions of the target cell. The plurality of double-stranded repair nucleic acid (dsDNA) molecules in the composition can alternatively target an order of 10³ to 10⁵ distinct target regions of the target cell, for example for synthetic biology engineering of a cellular system.

The target cell can be a mammalian cell, a bacterial cell, or an yeast cell. For select therapeutic embodiments, the target cell is a human cell.

The disclosure further contemplates synthetic linear constructs encoding the double-stranded repair nucleic acid (dsDNA) molecules comprising the transcriptional roadblocks described herein and vectors encoding the same.

Also contemplated by the disclosure are processes for homologous recombination-based gene editing at a transcriptional roadblock with the compositions of the disclosure. In some embodiments, the process comprises: introducing into a cell a double-stranded repair nucleic acid (dsDNA) cassette having a sequence encoding a guide RNA (gRNA) for recruiting an endonuclease; a sequence homologous to a target region of a target cell, wherein the sequence homologous to the target region has at least one nucleic acid base variation compared to the target region of the target cell; one or more transcriptional roadblock moiety(ies) at a transcriptional roadblock within a distance of a putative double-strand cleavage site for the endonuclease; and allowing the cell to grow under conditions that support: double-strand cleavage of a putative double-strand break target site by the endonuclease; and homologous recombination of a region of the double-stranded repair nucleic acid (dsDNA) cassette at the target region of the target cell.

In some cases, the disclosure provides a process for homologous recombination-based gene editing at a transcriptional roadblock comprising; introducing into a cell a single stranded repair nucleic acid (ssDNA repair) whereby the ssDNA repair nucleic acid is tethered via transcribed RNA to a double-stranded repair nucleic acid (dsDNA) editing cassette by an RNA polymerase (RNAP) molecule stalled at a transcriptional roadblock; and allowing the cell to grow under conditions that support double-strand cleavage of a putative double-strand break target site by the endonuclease; and homologous recombination of a region of the double-stranded repair nucleic acid (dsDNA) editing cassette at the target region of the target cell. The dsDNA repair nucleic acid editing cassette preferably has both a sequence encoding a guide RNA (gRNA) for recruiting an endonuclease and a sequence homologous to a target region of a target cell (e.g., repair DNA), wherein the sequence homologous to the target region has at least one nucleic acid base variation compared to the target region of the target cell.

In preferred embodiments gene expression of the sequence encoding the editing cassette comprising both the guide RNA and repair DNA is under control of an inducible promoter for greater control of the gene editing process. In some cases, the same inducible promoter drives transcription of both the editing cassette and the nuclease and activation of the inducible promoter drives the expression of double stranded repair nucleic acid (dsDNA repair) (e.g., editing cassette) comprising the gRNA and the sequence homologous to the target region. The disclosure contemplates a process whereby the gRNA recruits the endonuclease to the putative double-strand break target site, and effectively cleaves the same.

The disclosure further contemplates processes where the transcriptional roadblock is generated in an automated multi-module cell editing instrument, such as an automated multi-module cell editing instrument comprising a transformation module configured to introduce the editing cassette (and, in some embodiments, the coding sequence for the nuclease) into a plurality of cells. Exemplary automated multi-module instruments for the editing of live cells with transcriptional roadblocks of the disclosure are shown in FIG. 2A – FIG. 2C; and FIG. 3A to FIG. 3E. In some cases, the automated multi-module cell editing instrument further comprises a singulation assembly for substantially singulating the cell(s) that receives the editing cassette. In some instances, the at least one nucleic acid base variation is a deletion of a nucleobase, an addition of a nucleobase, or a replacement of a nucleobase compared to the target region of the target cell, which may optionally be designed to introduce a silent mutation on the target cell. In some cases, the endonuclease can be selected from the group consisting of MAD7, Cas9, or Cas12. Further wherein the double-stranded repair nucleic acid (dsDNA) molecule can comprise a site conferring immunity to further editing by the nuclease, such as a change in a PAM sequence for the nuclease. The target cell can be a mammalian cell, a bacterial cell, or an yeast cell, such as human cell.

Also disclosed herein is a process for generating a transcriptional roadblock that provides a local concentration of a repair nucleic acid (DNA repair) at a double-stranded template break site (dsDNA template break site) in a population of cells by: introducing a double-stranded repair nucleic acid cassette (dsDNA cassette or editing cassette) into at least one cell in the population of cells, the dsDNA cassette having a sequence encoding a guide RNA for recruiting an endonuclease, a sequence homologous to a target region of a target cell, wherein the sequence homologous to the target region has at least one nucleic acid base variation compared to the target region of the target cell, and one or more transcriptional roadblock moiety(ies) at a transcriptional roadblock within a distance of a putative double-strand cleavage site for the endonuclease; allowing the cell to grow under conditions that support transcription of the sequence encoding the guide RNA for recruiting the endonuclease from the dsDNA cassette and the sequence homologous to the target region of a target cell (e.g., repair DNA) by an endogenous RNA polymerase within the cell thus generating a ssDNA repair strand functionally connected to the endogenous RNA polymerase, whereby the endogenous RNA polymerase becomes stalled at the one or more transcription roadblocks in the cell providing the local concentration of the repair strand within 500 bases from the putative double-strand cleavage site for the endonuclease.

In some cases, the transcriptional roadblock is generated in an automated multi-module cell editing instrument, such as an instrument comprising a singulation assembly for substantially singulating the population of cells. The instrument can comprise a singulation assembly module for a solid wall isolation, incubation and normalization (SWIIN) comprising: a retentate member comprising at least one retentate port fluidically connected to a channel; a permeate member fluidically connected to the channel; a perforated member; a filter disposed under and adjacent to the perforated member and above and adjacent to the permeate member; and a gasket surrounding the filter.

These aspects and other features and advantages of the invention are described below in more detail.

### BRIEF DESCRIPTION OF THE DRAWINGS

The foregoing and other features and advantages of the present invention will be more fully understood from the following detailed description of illustrative embodiments taken in conjunction with the accompanying drawings in which:

FIG. 1A is a diagram illustrating an editing cassette tethered by an RNA polymerase (RNAP) molecule stalled at a transcriptional roadblock located at the 3′ end of the arms of homology of the dsDNA repair nucleic acid to a nuclease via binding of the nuclease to RNA transcribed from the dsDNA repair nucleic acid.

FIG. 1B is a diagram illustrating a dsDNA repair nucleic acid tethered by an RNA polymerase (RNAP) molecule stalled at a transcriptional roadblock within a coding region of the (dsDNA repair nucleic acid to a nuclease via binding of the nuclease to RNA transcribed from the dsDNA repair nucleic acid.

FIG. 1C is a diagram illustrating a biotin (circle with diagonal lines) -streptavidin (solid circle) transcriptional roadblock moiety engineered at a dsDNA repair nucleic acid. Upon transfection into a target cell, an RNA polymerase starts transcription at the J23119 promoter and transcribes a gRNA (crRNA and trRNA linked sequences) as well as the sequence homologous to a target region of the target cell (e.g., repair DNA) up until it reaches the transcriptional roadblock, when it becomes stalled within the cell.

FIG. 1D illustrates a characterization of an RNAP roadblock complex formation via PAGE analysis.

FIGS. 2A - 2C depict three different views of an exemplary automated multi-module cell processing instrument for performing nucleic acid-guided nuclease editing.

FIGS. 3A - 3C depict various components of exemplary embodiments of a bioreactor module included in an integrated instrument useful for growing and transfecting cells.

FIGS. 3D and 3E depict an exemplary integrated instrument for growing and transfecting cells.

It should be understood that the drawings are not necessarily to scale, and that like reference numbers refer to like features.

### DETAILED DESCRIPTION

All of the functionalities described in connection with one embodiment of the methods, devices or instruments described herein are intended to be applicable to the additional embodiments of the methods, devices and instruments described herein except where expressly stated or where the feature or function is incompatible with the additional embodiments. For example, where a given feature or function is expressly described in connection with one embodiment but not expressly mentioned in connection with an alternative embodiment, it should be understood that the feature or function may be deployed, utilized, or implemented in connection with the alternative embodiment unless the feature or function is incompatible with the alternative embodiment.

During transcription, cellular RNA polymerases (RNAPs) have to deal with numerous potential roadblocks imposed by various DNA binding proteins. Many such proteins partially or completely interrupt a single round of RNA chain elongation in vitro, a phenomenon believed to occur in many bacterial, yeast, and mammalian systems. Because transcription is so easily perturbed, DNA binding proteins, misincorporated nucleotides and other errors are quite frequent and can cause the RNAP enzyme to arrest. In such a case, the polymerase may move in retrograde, sliding a short distance in the opposite direction along the DNA, so that the defect can be repaired. In some instances however, this may bring the transcription process to a complete halt and cause the RNAP to remain stalled at a template.

The present disclosure contemplates the formation of transcriptional roadblocks that favor the stalling of an RNAP at a transcriptional roadblock. Specifically, the disclosure contemplates the engineering of nucleic acid sequences encoding various gRNAs and repair DNAs at a location sufficiently close to a putative transcriptional roadblock. The templates of the disclosure-preferably double-stranded cassettes—are transcribed by an RNAP up until the RNAP reaches a transcriptional roadblock. Upon reaching the transcriptional roadblock, the stalled RNAP remains functionally tethered to a gRNA and a repair nucleic acid sequence for use in a homology directed repair (HDR) pathway by a cell.

Homology directed repair (HDR) pathways can occur either non-conservatively or conservatively. The non-conservative method is composed of the single-strand annealing (SSA) pathway and is more error prone. The conservative methods, characterized by repair of the DSB by means of a “template” homologous repair DNA (e.g., sister chromatid, plasmid, etc.) is often more precise. In the classical double-strand break repair (DSBR) pathway, the 3′ ends invade an intact homologous template to serve as a primer for DNA repair synthesis, ultimately leading to the formation of double Holliday junctions (dHJs). dHJs are four-stranded branched structures that form when elongation of the invasive strand “captures” and synthesizes DNA from the second DSB end. In the synthesis-dependent strand-annealing (SDSA) pathway, unlike DSBR, following strand invasion and D loop formation in SDSA, the newly synthesized portion of the invasive strand is displaced from the template and returned to the processed end of the non-invading strand at the other DSB end. The 3′ end of the non-invasive strand is elongated and ligated to fill the gap, thus completing SDSA.

The disclosure provides editing cassettes, plasmids, and vectors that employ homologous recombination for genome engineering by having a CRISPR nuclease cause a specific DSB, while tethered to a repair repair nucleic acid. HDR templates used to create specific mutations or insert new elements into a gene require a certain amount of homology surrounding the target sequence that will be modified. Generally, the disclosure contemplates homology arms that start at the CRISPR-nuclease induced DSB. Typically, the insertion sites of the modification should be very close to the DSB, such as within 500 bases, within 250 bases within 100 bases, within 50 bases, or within 10 bases or less of the putative double-strand cleavage site. The disclosure contemplates a transcriptional roadblock that itself is within 500 bases, within 250 bases, within 100 bases, or within 50 bases of the putative double-strand cleavage site.

Further, the CRISPR nucleases may continue to cleave the target nucleic acid once a DSB is introduced and repaired. As long as the gRNA target site/PAM site remain intact, the endonuclease may keep cutting and repairing the DNA. This repeated editing is counter to the goal of introducing a very specific mutation or sequence in the target region. To overcome this challenge, the disclosure contemplates double-stranded repair nucleic acid (dsDNA) cassettes that will ultimately block further nuclease targeting after the initial DSB is repaired. The disclosure contemplates blocking further editing by designing cassettes that have at least one nucleic acid base variation compared to the target region of the target cell. In some cases the variation is designed to introduce a silent mutation on the target cell. The silent mutation may provide a site conferring immunity to further editing by the nuclease, for example by having a change in a PAM sequence for the nuclease.

Notably, the disclosure contemplates the generation of transcription roadblocks that are assembled in vivo within a cell, as well as transcriptional roadblocks that are pre-assembled in vitro prior to administration to a cell. For instance, a dsDNA cassette can be engineered to have a ligand at a transcriptional roadblock pre-determined site, such as a biotin molecule. The dsDNA cassette can be transfected into a cell that is grown in the presence of a ligand binding moiety such as avidin/streptavidin. In such cases, the transcriptional roadblock may be assembled in vivo within a cell. Alternatively, the transcriptional roadblock can be a non-canonical nucleobase within the cassette, such as nucleobases known to create stalling of RNAP, for example 5-guanidinohydantoin (Gh) and spiroiminodihydantoin (Sp). In other cases, the transcriptional roadblock may be pre-assembled in vitro prior to transfection into a cell.

The practice of the techniques described herein may employ, unless otherwise indicated, conventional techniques and descriptions of molecular biology (including recombinant techniques), cell biology, biochemistry, and genetic engineering technology, which are within the skill of those who practice in the art. Such conventional techniques and descriptions can be found in standard laboratory manuals such as Green and Sambrook, Molecular Cloning: A Laboratory Manual. 4th ed., Cold Spring Harbor Laboratory Press, Cold Spring Harbor, N.Y., (2014); Current Protocols in Molecular Biology, Ausubel, et al. eds., (2017); Neumann, et al., Electroporation and Electrofusion in Cell Biology, Plenum Press, New York (1989); Chang, et al., Guide to Electroporation and Electrofusion, Academic Press, California (1992); Viral Vectors (Kaplift & Loewy, eds., Academic Press (1995)); all of which are herein incorporated in their entirety by reference for all purposes. For mammalian/stem cell culture and methods see, e.g., Basic Cell Culture Protocols, 4th ed. (Helgason & Miller, eds., Humana Press 2005); Culture of Animal Cells, Seventh Ed. (Freshney, ed., Humana Press 2016); Microfluidic Cell Culture, Second Ed. (Borenstein, Vandon, Tao & Charest, eds., Elsevier Press 2018); Human Cell Culture (Hughes, ed., Humana Press 2011); 3D Cell Culture (Koledova, ed., Humana Press 2017); Cell and Tissue Culture: Laboratory Procedures in Biotechnology (Doyle & Griffiths, eds., John Wiley & Sons 1998); Essential Stem Cell Methods, (Lanza & Klimanskaya, eds., Academic Press 2011); Essentials of Stem Cell Biology, 3d ed., (Lanza & Atala, eds., Academic Press 2013); and Handbook of Stem Cells, (Atala & Lanza, eds., Academic Press 2012), all of which are herein incorporated in their entirety by reference for all purposes. CRISPR editing techniques can be found in, e.g., Genome Editing and Engineering from TALENs and CRISPRs to Molecular Surgery, Appasani and Church (2018); and CRISPR: Methods and Protocols, Lindgren and Charpentier (2015); both of which are herein incorporated in their entirety by reference for all purposes.

Note that as used herein and in the appended claims, the singular forms “a,” “an,” and “the” include plural referents unless the context clearly dictates otherwise. Thus, for example, reference to “a cell” refers to one or more cells, and reference to “the system” includes reference to equivalent steps, methods and devices known to those skilled in the art, and so forth. Additionally, it is to be understood that terms such as “left,” “right,” “top,” “bottom,” “front,” “rear,” “side,” “height,” “length,” “width,” “upper,” “lower,” “interior,” “exterior,” “inner,” “outer” that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Unless defined otherwise, all technical and scientific terms used herein have the same meaning as commonly understood by one of ordinary skill in the art to which this invention belongs. All publications mentioned herein are incorporated by reference for the purpose of describing and disclosing devices, formulations and methodologies that may be used in connection with the presently described invention.

Where a range of values is provided, it is understood that each intervening value, between the upper and lower limit of that range and any other stated or intervening value in that stated range is encompassed within the invention. The upper and lower limits of these smaller ranges may independently be included in smaller ranges, and are also encompassed within the invention, subject to any specifically excluded limit in the stated range. Where the stated range includes one or both of the limits, ranges excluding either or both of those included limits are also included in the invention.

In the following description, numerous specific details are set forth to provide a more thorough understanding of the present invention. However, it will be apparent to one of skill in the art that the present invention may be practiced without one or more of these specific details. In other instances, features and procedures well known to those skilled in the art have not been described in order to avoid obscuring the invention. The terms used herein are intended to have the plain and ordinary meaning as understood by those of ordinary skill in the art.

The term “transcriptional roadblock” collectively refers to complexes formed by cellular RNA polymerases (RNAPs) stalled at intrinsic or extrinsic obstacles at a double-stranded repair nucleic acid (dsDNA) cassette. The extrinsic obstacles can be moieties physically blocking RNAP transcription (e.g., biotin/streptavidin) or intrinsic obstacles such as non-canonical nucleobases.

The term “non-canonical nucleobase” refers to one nucleobase or a stretch of nucleobases capable of stalling the progression of an RNA polymerase, such as 5-guanidinohydantoin (Gh) and spiroiminodihydantoin (Sp). See, e.g., RNA polymerase II stalls on oxidative DNA damage via a torsion-latch mechanism involving lone pair-π and CH-π interactions, PNAS Apr. 28, 2020 117 (17) 9338-9348.

The term “complementary” as used herein refers to Watson-Crick base pairing between nucleotides and specifically refers to nucleotides hydrogen bonded to one another with thymine or uracil residues linked to adenine residues by two hydrogen bonds and cytosine and guanine residues linked by three hydrogen bonds. In general, a nucleic acid includes a nucleotide sequence described as having a “percent complementarity” or “percent homology” to a specified second nucleotide sequence. For example, a nucleotide sequence may have 80%, 90%, or 100% complementarity to a specified second nucleotide sequence, indicating that 8 of 10, 9 of 10 or 10 of 10 nucleotides of a sequence are complementary to the specified second nucleotide sequence. For instance, the nucleotide sequence 3′-TCGA-5′ is 100% complementary to the nucleotide sequence 5′-AGCT-3′; and the nucleotide sequence 3′-TCGA-5′ is 100% complementary to a region of the nucleotide sequence 5′-TAGCTG-3′.

The term DNA “control sequences” refers collectively to promoter sequences, polyadenylation signals, transcription termination sequences, upstream regulatory domains, origins of replication, internal ribosome entry sites, nuclear localization sequences, enhancers, and the like, which collectively provide for the replication, transcription and translation of a coding sequence in a recipient cell. Not all of these types of control sequences need to be present so long as a selected coding sequence is capable of being replicated, transcribed and-for some components—translated in an appropriate host cell.

The terms “editing cassette”, “CREATE cassette”, “CREATE editing cassette”, “CREATE fusion editing cassette” or “CFE editing cassette” refers to a nucleic acid molecule comprising a coding sequence for transcription of a guide nucleic acid or gRNA covalently linked to a coding sequence for transcription of a repair template.

As used herein, “enrichment” refers to enriching for edited cells by singulation, inducing editing, and growth of singulated cells into terminal-sized colonies (e.g., saturation or normalization of colony growth).

The terms “guide nucleic acid” or “guide RNA” or “gRNA” refer to a polynucleotide comprising 1) a guide sequence capable of hybridizing to a genomic target locus, and 2) a scaffold sequence capable of interacting or complexing with a nucleic acid-guided nuclease. The term “editing gRNA” refers to the gRNA used to edit a target sequence in a cell, typically a sequence endogenous to the cell.

“Homology” or “identity” or “similarity” refers to sequence similarity between two peptides or, more often in the context of the present disclosure, between two nucleic acid molecules. The term “homologous region” or “homology arm” refers to a region on the repair template with a certain degree of homology with the target genomic DNA sequence. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences.

“Operably linked” refers to an arrangement of elements where the components so described are configured so as to perform their usual function. Thus, control sequences operably linked to a coding sequence are capable of effecting the transcription, and in some cases, the translation, of a coding sequence. The control sequences need not be contiguous with the coding sequence so long as they function to direct the expression of the coding sequence. Thus, for example, intervening untranslated yet transcribed sequences can be present between a promoter sequence and the coding sequence and the promoter sequence can still be considered “operably linked” to the coding sequence. In fact, such sequences need not reside on the same contiguous DNA molecule (i.e. chromosome) and may still have interactions resulting in altered regulation.

A “promoter” or “promoter sequence” is a DNA regulatory region capable of binding RNA polymerase and initiating transcription of a polynucleotide or polypeptide coding sequence such as messenger RNA, ribosomal RNA, small nuclear or nucleolar RNA, guide RNA, or any kind of RNA transcribed by any class of any RNA polymerase I, II or III. Promoters may be constitutive or inducible.

As used herein, the terms “protein” and “polypeptide” are used interchangeably. “Recognition sequences” are particular sequences of nucleotides that a protein, DNA, or RNA molecule, or combinations thereof (such as, but not limited to, a restriction endonuclease, a modification methylase or a recombinase) recognizes and binds. For example, a recognition sequence for Cre recombinase is a 34 base pair sequence containing two 13 base pair inverted repeats (serving as the recombinase binding sites) flanking an 8 base pair core and designated loxP (see, e.g., Sauer, Current Opinion in Biotechnology, 5:521-527 (1994)). Other examples of recognition sequences include, but are not limited to, attB and attP, attR and attL and others that are recognized by the recombinase enzyme bacteriophage Lambda Integrase. The recombination site designated attB is an approximately 33 base pair sequence containing two 9 base pair core-type Int binding sites and a 7 base pair overlap region; attP is an approximately 240 base pair sequence containing core-type Int binding sites and arm-type Int binding sites as well as sites for auxiliary proteins IHF, FIS, and Xis (see, e.g., Landy, Current Opinion in Biotechnology, 3:699-7071 (1993)).

A “recombinase” is an enzyme that catalyzes the exchange of DNA segments at specific recombination sites. An “integrase” refers to a recombinase that is usually derived from viruses or transposons, as well as perhaps ancient viruses. “Recombination proteins” include excisive proteins, integrative proteins, enzymes, co-factors and associated proteins that are involved in recombination reactions using one or more recombination sites (again see, e.g., Landy, Current Opinion in Biotechnology, 3:699-707 (1993)). The recombination proteins used in the methods herein can be delivered to a cell via an expression cassette on an appropriate vector, such as a plasmid or viral vector. In other embodiments, recombination proteins can be delivered to a cell in protein form in the same reaction mixture used to deliver the desired nucleic acid(s). In yet other embodiments, the recombinase could also be encoded in the cell and expressed upon demand using a tightly controlled inducible promoter.

As used herein the terms “repair template” or “donor nucleic acid” or “donor DNA” or “homology arm” refer to 1) nucleic acid that is designed to introduce a DNA sequence modification (insertion, deletion, substitution) into a locus by homologous recombination using nucleic acid-guided nucleases, or 2) a nucleic acid that serves as a template (including a desired edit) to be incorporated into target DNA by reverse transcriptase in a CREATE TRANSCRIPTIONAL ROADBLOCK system. For homology-directed repair, the repair template must have sufficient homology to the regions flanking the “cut site” or the site to be edited in the genomic target sequence. For template-directed repair, the repair template has homology to the genomic target sequence except at the position of the desired edit although synonymous edits may be present in the homologous (e.g., non-edit) regions. In many instances and preferably, the repair template will have two regions of sequence homology (e.g., two homology arms) complementary to the genomic target locus flanking the locus of the desired edit in the genomic target locus. Typically, an “edit region” or “edit locus” or “DNA sequence modification” region-the nucleic acid modification that one desires to be introduced into a genome target locus in a cell (e.g., the desired edit)-will be located between two regions of homology and a target sequence. A deletion or insertion may be a deletion or insertion of 1, 2, 3, 4, 5, 10, 15, 20, 25, 30, 40, 50, 75, 100, 150, 200, 300, 400, or 500 or more base pairs of the target sequence.

As used herein the term “selectable marker” refers to a gene introduced into a cell, which confers a trait suitable for artificial selection. General use selectable markers are well-known to those of ordinary skill in the art. Drug selectable markers such as ampicillin/carbenicillin, kanamycin, nourseothricin N-acetyl transferase, chloramphenicol, erythromycin, tetracycline, gentamicin, bleomycin, streptomycin, rifampicin, puromycin, hygromycin, blasticidin, and G418 may be employed. In other embodiments, selectable markers include, but are not limited to sugars such as rhamnose, human nerve growth factor receptor (detected with a MAb, such as described in U.S. Pat. No. 6,365,373); truncated human growth factor receptor (detected with MAb); mutant human dihydrofolate reductase (DHFR; fluorescent MTX substrate available); secreted alkaline phosphatase (SEAP; fluorescent substrate available); human thymidylate synthase (TS; confers resistance to anti-cancer agent fluorodeoxyuridine); human glutathione S-transferase alpha (GSTA1; conjugates glutathione to the stem cell selective alkylator busulfan; chemoprotective selectable marker in CD34+cells); CD24 cell surface antigen in hematopoietic stem cells; human CAD gene to confer resistance to N-phosphonacetyl-L-aspartate (PALA); human multi-drug resistance-1 (MDR-1; P-glycoprotein surface protein selectable by increased drug resistance or enriched by FACS); human CD25 (IL-2α; detectable by Mab-FITC); Methylguanine-DNA methyltransferase (MGMT; selectable by carmustine); rhamnose; and Cytidine deaminase (CD; selectable by Ara-C). “Selective medium” as used herein refers to cell growth medium to which has been added a chemical compound or biological moiety that selects for or against selectable markers.

The term “specifically binds” as used herein includes an interaction between two molecules, e.g., a transcriptional roadblock ligand and a transcriptional roadblock ligand-binding moiety, with a binding affinity represented by a dissociation constant of about 10⁻⁷ M, about 10⁻⁸ M, about 10⁻⁹ M, about 10⁻¹⁰ M, about 10⁻¹¹ M, about 10⁻¹² M, about 10⁻¹³ M, about 10⁻¹⁴ M or about 10⁻¹⁵ M.

The terms “target genomic DNA sequence”, “cellular target sequence”, or “genomic target locus” and the like refer to any locus in vitro or in vivo, or in a nucleic acid (e.g., genome or episome) of a cell or population of cells, in which a change of at least one nucleotide is desired using a nucleic acid-guided nuclease editing system. The cellular target sequence can be a genomic locus or extrachromosomal locus.

The terms “transformation”, “transfection” and “transduction” are used interchangeably herein to refer to the process of introducing exogenous DNA into cells.

The term “variant” may refer to a polypeptide or polynucleotide that differs from a reference polypeptide or polynucleotide but retains essential properties. A typical variant of a polypeptide differs in amino acid sequence from another reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A variant of a polypeptide may be a conservatively modified variant. A substituted or inserted amino acid residue may or may not be one encoded by the genetic code (e.g., a non-natural amino acid). A variant of a polypeptide may be naturally occurring, such as an allelic variant, or it may be a variant that is not known to occur naturally.

A “vector” is any of a variety of nucleic acids that comprise a desired sequence or sequences to be delivered to and/or expressed in a cell. Vectors are typically composed of DNA, although RNA vectors are also available. Vectors include, but are not limited to, plasmids, fosmids, phagemids, virus genomes, BACs, YACs, PACs, synthetic chromosomes, and the like.

As used herein, the phrase “engine vector” comprises a coding sequence for a nuclease to be used in the nucleic acid-guided nuclease systems and methods of the present disclosure. The engine vector may also comprise, in a bacterial system, the λ Red recombineering system or an equivalent thereof. Engine vectors also typically comprise a selectable marker. As used herein the phrase “editing vector” comprises a repair nucleic acid, including an alteration to the cellular target sequence that prevents nuclease binding at a PAM or spacer in the cellular target sequence after editing has taken place, and a coding sequence for a gRNA. The editing vector may also and preferably does comprise a selectable marker and/or a barcode. In some embodiments, the engine vector and editing vector may be combined; that is, all editing and selection components may be found on a single vector. Further, the engine and editing vectors comprise control sequences operably linked to, e.g., the nuclease coding sequence, recombineering system coding sequences (if present), repair nucleic acid, guide nucleic acid(s), and selectable marker(s).

### Nuclease-Directed Genome Editing Generally

In preferred embodiments, the automated instrument described herein performs nuclease-directed genome editing methods for introducing edits to a population of cells. A recent discovery for editing live cells involves nucleic acid-guided nuclease (e.g., RNA-guided nuclease) editing. A nucleic acid-guided nuclease complexed with an appropriate synthetic guide nucleic acid in a cell can cut the genome of the cell at a desired location. The guide nucleic acid helps the nucleic acid-guided nuclease recognize and cut the DNA at a specific target sequence. By manipulating the nucleotide sequence of the guide nucleic acid, the nucleic acid-guided nuclease may be programmed to target any DNA sequence for cleavage as long as an appropriate protospacer adjacent motif (PAM) is nearby. In certain aspects, the nucleic acid-guided nuclease editing system may use two separate guide nucleic acid molecules that combine to function as a guide nucleic acid, e.g., a CRISPR RNA (crRNA) and trans-activating CRISPR RNA (tracrRNA). In other aspects, the guide nucleic acid may be a single guide nucleic acid that includes both the crRNA and tracrRNA sequences.

In general, a guide nucleic acid (e.g., gRNA) complexes with a compatible nucleic acid-guided nuclease and can then hybridize with a target sequence, thereby directing the nuclease to the target sequence. A guide nucleic acid can be DNA or RNA; alternatively, a guide nucleic acid may comprise both DNA and RNA. In some embodiments, a guide nucleic acid may comprise modified or non-naturally occurring nucleotides. In cases where the guide nucleic acid comprises RNA, the gRNA may be encoded by a DNA sequence on a polynucleotide molecule such as a plasmid, linear construct, or the coding sequence may and preferably does reside within an editing cassette and is under the control of an inducible promoter as described below. The present disclosure provides “CREATE TRANSCRIPTIONAL ROADBLOCK” editing cassettes and libraries of that are alternatives to traditional “CREATE” editing cassettes, see U.S.P.Ns 9,982,278; 10,266,849; 10,240,167; 10,351,877; 10,364,442; 10,435,715; 10,465,207; 10,669,559; 10,711,284; 11,078,498; 10,731,180; and U.S.S.Ns 16/550,092 and 17/222,936; all of which are incorporated by reference herein.

A guide nucleic acid comprises a guide sequence, where the guide sequence is a polynucleotide sequence having sufficient complementarity with a target sequence to hybridize with the target sequence and direct sequence-specific binding of a complexed nucleic acid-guided nuclease to the target sequence. The degree of complementarity between a guide sequence and the corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences. In some embodiments, a guide sequence is about or more than about 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20 nucleotides in length. Preferably the guide sequence is 10-30 or 15-20 nucleotides long, or 15, 16, 17, 18, 19, or 20 nucleotides in length.

In the present methods and compositions, the guide nucleic acids are provided as a sequence to be expressed from a plasmid or vector and comprises both the guide sequence and the scaffold sequence as a single transcript under the control of an inducible promoter. The guide nucleic acids are engineered to target a desired target sequence (either cellular target sequence or curing target sequence) by altering the guide sequence so that the guide sequence is complementary to a desired target sequence, thereby allowing hybridization between the guide sequence and the target sequence. In general, to generate an edit in the target sequence, the gRNA/nuclease complex binds to a target sequence as determined by the guide RNA, and the nuclease recognizes a protospacer adjacent motif (PAM) sequence adjacent to the target sequence. The target sequence can be any polynucleotide endogenous or exogenous to a prokaryotic or eukaryotic cell, or in vitro. For example, the target sequence can be a polynucleotide residing in the nucleus of a eukaryotic cell. A target sequence can be a sequence encoding a gene product (e.g., a protein) or a non-coding sequence (e.g., a regulatory polynucleotide, an intron, a PAM, or “junk” DNA) or a curing target sequence in an editing vector. In the present description, the target sequence for one of the gRNAs, the curing gRNA, is on the editing vector.

The editing guide nucleic acid may be and preferably is part of an editing cassette that encodes the repair template that targets a cellular target sequence. Alternatively, the guide nucleic acid may not be part of the editing cassette and instead may be encoded on the editing vector backbone. For example, a sequence coding for a guide nucleic acid can be assembled or inserted into a vector backbone first, followed by insertion of the repair template in, e.g., an editing cassette. In other cases, the repair template in, e.g., an editing cassette can be inserted or assembled into a vector backbone first, followed by insertion of the sequence coding for the guide nucleic acid. Preferably, the sequence encoding the guide nucleic acid and the repair template are located together in a rationally-designed editing cassette and are simultaneously inserted or assembled via gap repair into a linear plasmid or vector backbone to create an editing vector. In yet other embodiments, the sequence encoding the guide nucleic acid and the sequence encoding the repair nucleic acid are both included in the editing cassette.

The target sequence is associated with a proto-spacer mutation (PAM), which is a short nucleotide sequence recognized by the gRNA/nuclease complex. The precise preferred PAM sequence and length requirements for different nucleic acid-guided nucleases vary; however, PAMs typically are 2-10 or so base-pair sequences adjacent or in proximity to the target sequence and, depending on the nuclease, can be 5′ or 3′ to the target sequence. Engineering of the PAM-interacting domain of a nucleic acid-guided nuclease may allow for alteration of PAM specificity, improve target site recognition fidelity, decrease target site recognition fidelity, or increase the versatility of a nucleic acid-guided nuclease.

In most embodiments, genome editing of a cellular target sequence both introduces a desired DNA change to a cellular target sequence (an “intended” edit), e.g., the genomic DNA of a cell, and removes, mutates, or renders inactive a proto-spacer mutation (PAM) region in the cellular target sequence (an “immunizing edit”) thereby rendering the target site immune to further nuclease binding. Rendering the PAM at the cellular target sequence inactive precludes additional editing of the cell genome at that cellular target sequence, e.g., upon subsequent exposure to a nucleic acid-guided nuclease complexed with a synthetic guide nucleic acid in later rounds of editing. Thus, cells having the desired cellular target sequence edit and an altered PAM can be selected for by using a nucleic acid-guided nuclease complexed with a synthetic guide nucleic acid complementary to the cellular target sequence. Cells that did not undergo the first editing event will be cut rendering a double-stranded DNA break, and thus will not continue to be viable. The cells containing the desired cellular target sequence edit and PAM alteration will not be cut, as these edited cells no longer contain the necessary PAM site and will continue to grow and propagate.

As for the nuclease component of the nucleic acid-guided nuclease editing system, a polynucleotide sequence encoding the nucleic acid-guided nuclease can be codon optimized for expression in particular cell types, such as archaeal, prokaryotic or eukaryotic cells. Eukaryotic cells can be yeast, fungi, algae, plant, animal, or human cells. Eukaryotic cells may be those of or derived from a particular organism, such as a mammal, including but not limited to human, mouse, rat, rabbit, dog, or non-human mammals including non-human primates. The choice of nucleic acid-guided nuclease to be employed depends on many factors, such as what type of edit is to be made in the target sequence and whether an appropriate PAM is located close to the desired target sequence. CRISPR nucleases of use in the methods described herein include but are not limited to Cas 9, Cas 12/CpfI, MAD2, or MAD7, MAD 2007 or other MADzymes and MADzyme systems (see U.S.P.Ns 9,982,279; 10,337,028; 10,435,714; 10,011,849; 10,626,416; 10,604,746; 10,665,114; 10,640,754; 10,876,102; 10,883,077; 10,704,033; 10,745,678; 10,724,021; 10,767,169; and 10,870,761 for sequences and other details related to engineered and naturally-occuring MADzymes). Nuclease fusion enzymes typically comprise a CRISPR nucleic acid-guided nuclease engineered to cut one DNA strand in the target DNA rather than making a double-stranded cut, and the nuclease portion is fused to a reverse transcriptase. For more information on nickases and nuclease fusion editing see U.S.P.Ns 10,689,669; and U.S.P.Ps 20210214671. As with the guide nucleic acid, the nuclease is encoded by a DNA sequence on a vector (e.g., the engine vector) and be under the control of an inducible promoter. In some embodiments, the inducible promoter may be separate from but the same as the inducible promoter controlling transcription of the guide nucleic acid; that is, a separate inducible promoter drives the transcription of the nuclease or nuclease fusion and guide nucleic acid sequences but the two inducible promoters may be the same type of inducible promoter (e.g., both are pL promoters). Alternatively, the inducible promoter controlling expression of the nuclease may be different from the inducible promoter controlling transcription of the guide nucleic acid; that is, e.g., the nuclease may be under the control of the pBAD inducible promoter, and the guide nucleic acid may be under the control of the pL inducible promoter.

Another component of the nucleic acid-guided nuclease system is the repair template comprising homology to the cellular target sequence. For the present methods and compositions, the repair template typically is on the same vector and in the same editing cassette as the guide nucleic acid and is under the control of the same promoter as the editing gRNA (that is, a single promoter driving the transcription of both the editing gRNA and the repair template). The repair template is designed to serve as a template for homologous recombination with a cellular target sequence nicked or cleaved by the nucleic acid-guided nuclease as a part of the gRNA/nuclease complex. A repair template polynucleotide may be of any suitable length, such as about or more than about 20, 25, 50, 75, 100, 150, 200, 500, or 1000 nucleotides in length. In certain preferred aspects, the repair template can be provided as an oligonucleotide of between 20-100 nucleotides, more preferably between 30-75 nucleotides. The repair template comprises two regions that are complementary to a portion of the cellular target sequence (e.g., homology arms) flanking the mutation or difference between the repair template and the cellular target sequence. When optimally aligned, the repair template overlaps with (is complementary to) the cellular target sequence by, e.g., about 20, 25, 30, 35, 40, 50, 60, 70, 80, 90 or more nucleotides. The repair nucleic acid comprises two homology arms (regions complementary to the cellular target sequence) flanking the mutation or difference between the repair nucleic acid and the cellular target sequence. The repair nucleic acid comprises at least one mutation or alteration compared to the cellular target sequence, such as an insertion, deletion, modification, or any combination thereof compared to the cellular target sequence.

As described in relation to the gRNA, the repair template is provided as part of a rationally-designed editing cassette, which is inserted into an editing plasmid backbone where the editing plasmid backbone may comprise a promoter to drive transcription of the editing gRNA and the repair template when the editing cassette is inserted into the editing plasmid backbone. Moreover, there may be more than one, e.g., two, three, four, or more editing gRNA/repair template rationally-designed editing cassettes inserted into an editing vector targeting different regions of the genome; alternatively, a single rationally-designed editing cassette may comprise two to several editing gRNA/repair template pairs targeting different regions of the genome, where each editing gRNA is under the control of separate different promoters, separate like promoters, or where all gRNAs/ repair template pairs are under the control of a single promoter. In some embodiments the promoter driving transcription of the editing gRNA and the repair template (or driving more than one editing gRNA/ repair template pair) is optionally an inducible promoter.

Inducible editing is advantageous in that cells can be grown for several to many cell doublings before editing is initiated, which increases the likelihood that cells with edits will survive, as the double-strand cuts caused by active editing are largely toxic to the cells. This toxicity results both in cell death in the edited colonies, as well as possibly a lag in growth for the edited cells that do survive but must repair and recover following editing. However, once the edited cells have a chance to recover, the size of the colonies of the edited cells will eventually catch up to the size of the colonies of unedited cells. It is this toxicity, however, that is exploited herein to perform curing.

In addition to the repair template, an editing cassette may comprise one or more primer binding sites. The primer binding sites are used to amplify the editing cassette by using oligonucleotide primers; for example, if the primer sites flank one or more of the other components of the editing cassette.

Also, as described above, the repair nucleic acid may comprise-in addition to the at least one mutation relative to a cellular target sequence—one or more PAM sequence alterations that mutate, delete or render inactive the PAM site in the cellular target sequence. The PAM sequence alteration in the cellular target sequence renders the PAM site “immune” to the nucleic acid-guided nuclease and may be biotinylated or otherwise labeled.

In addition, the editing cassette may comprise a barcode. A barcode is a unique DNA sequence that corresponds to the repair DNA sequence such that the barcode can identify the edit made to the corresponding cellular target sequence. The barcode typically comprises four or more nucleotides. In some embodiments, the editing cassettes comprise a collection or library editing gRNAs and of repair nucleic acids representing, e.g., gene-wide or genome-wide libraries of editing gRNAs and repair nucleic acids. The library of editing cassettes is cloned into vector backbones where, e.g., each different repair nucleic acid is associated with a different barcode.

Additionally, in preferred embodiments, an editing vector or plasmid encoding components of the nucleic acid-guided nuclease system further encodes a nucleic acid-guided nuclease comprising one or more nuclear localization sequences (NLSs), such as about or more than about 1, 2, 3, 4, 5, 6, 7, 8, 9, 10, or more NLSs, particularly as an element of the nuclease sequence. In some embodiments, the engineered nuclease comprises NLSs at or near the amino-terminus, NLSs at or near the carboxy-terminus, or a combination.

The engine and editing vectors comprise control sequences operably linked to the component sequences to be transcribed. As stated above, the promoters driving transcription of one or more components of the nucleic acid-guided nuclease editing system preferably are inducible. A number of gene regulation control systems have been developed for the controlled expression of genes in plant, microbe, and animal cells, including mammalian cells, including the pL promoter (induced by heat inactivation of the cI857 repressor), the pPhIF promoter (induced by the addition of 2,4 diacetylphloroglucinol (DAPG)), the pBAD promoter (induced by the addition of arabinose to the cell growth medium), and the rhamnose inducible promoter (induced by the addition of rhamnose to the cell growth medium). Other systems include the tetracycline-controlled transcriptional activation system (Tet-On/Tet-Off, Clontech, Inc. (Palo Alto, CA); Bujard and Gossen, PNAS, 89(12):5547-5551 (1992)), the Lac Switch Inducible system (Wyborski et al., Environ Mol Mutagen, 28(4):447-58 (1996); DuCoeur et al., Strategies 5(3):70-72 (1992); U.S. Pat. No. 4,833,080), the ecdysone-inducible gene expression system (No et al., PNAS, 93(8):3346-3351 (1996)), the cumate gene-switch system (Mullick et al., BMC Biotechnology, 6:43 (2006)), and the tamoxifen-inducible gene expression (Zhang et al., Nucleic Acids Research, 24:543-548 (1996)) as well as others. In the present methods used in the modules and instruments described herein, it is preferred that at least one of the nucleic acid-guided nuclease editing components (e.g., the nuclease and/or the gRNA) is under the control of a promoter that is activated by a rise in temperature, as such a promoter allows for the promoter to be activated by an increase in temperature, and de-activated by a decrease in temperature, thereby “turning off” the editing process. Thus, in the scenario of a promoter that is de-activated by a decrease in temperature, editing in the cell can be turned off without having to change media; to remove, e.g., an inducible biochemical in the medium that is used to induce editing.

### RNAP Roadblocks

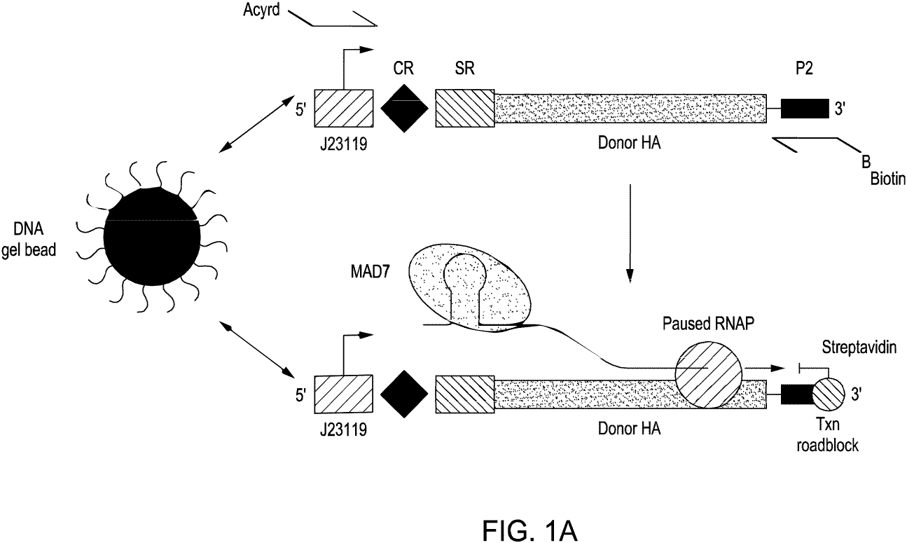

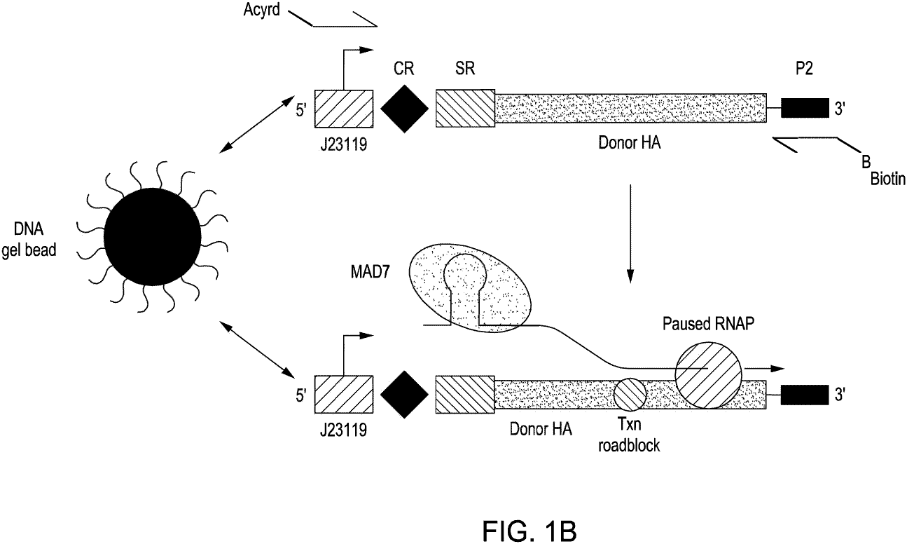

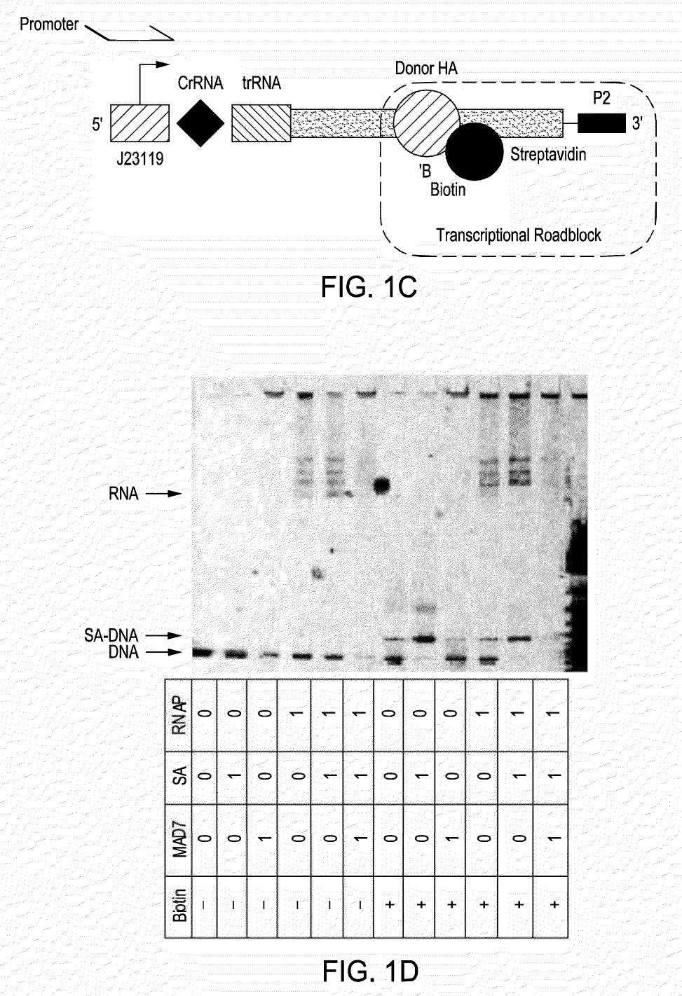

FIG. 1A is a diagram illustrating a dsDNA repair nucleic acid tethered by an RNA polymerase (RNAP) molecule stalled at a transcriptional roadblock located at the 3′ end of the arms of homology of the dsDNA repair DNA to a nuclease via binding of the nuclease to RNA transcribed from the editing cassette. FIG. 1B is a diagram illustrating a dsDNA repair nucleic acid tethered by an RNA polymerase (RNAP) molecule stalled at a transcriptional roadblock within a coding region of the dsDNA repair nucleic acid to a nuclease via binding of the nuclease to RNA transcribed from the editing cassette. FIG. 1C is a diagram illustrating a biotin (circle with diagonal lines) – streptavidin (solid circle) transcriptional roadblock moiety engineered at a dsDNA repair nucleic acid. Upon transfection into a target cell, an RNA polymerase starts transcription at the J23119 promoter and transcribes a gRNA (crRNA and trRNA linked sequences) as well as the repair DNA up until it reaches the transcriptional roadblock, when it becomes stalled within the cell.

Once designed and synthesized, the editing cassettes described in FIG. 1A -FIG. 1C are used in a process for homologous recombination-based gene editing comprising: introducing into a cell a double-stranded repair nucleic acid (dsDNA) cassette of FIG. 1A – FIG. 1C, and allowing the cell to grow under conditions that 1) support double-strand cleavage of a putative double-strand break target site by the endonuclease and permit homologous recombination of a region of the double-stranded repair nucleic acid (dsDNA) cassette at the target region of the target cell.

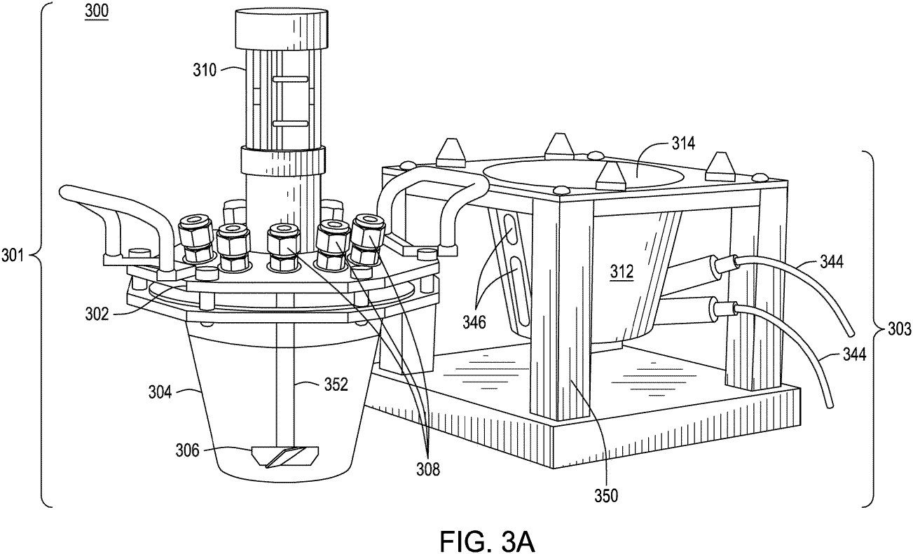

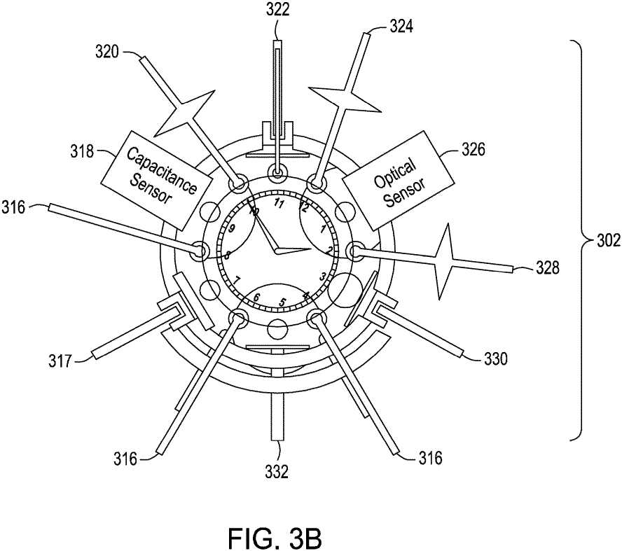

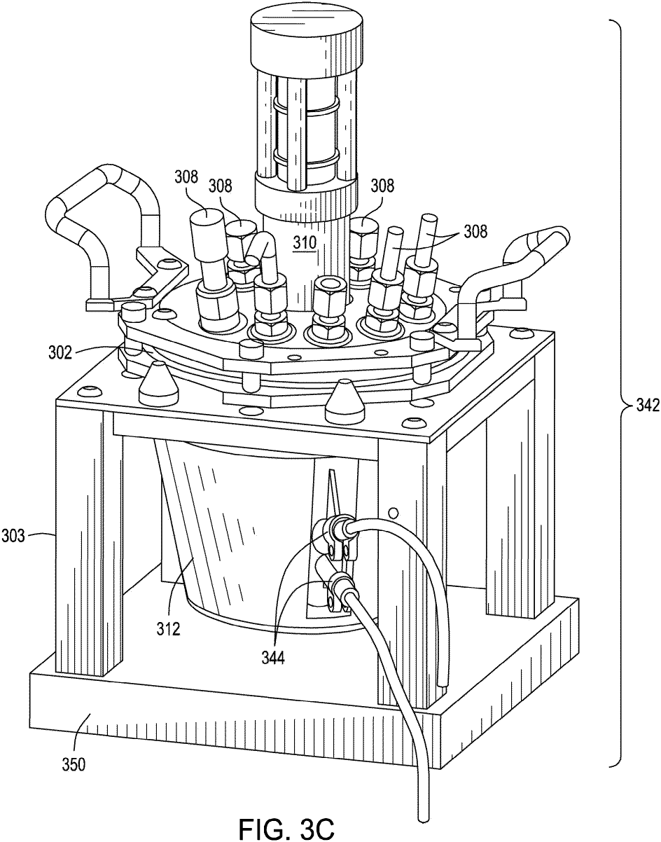

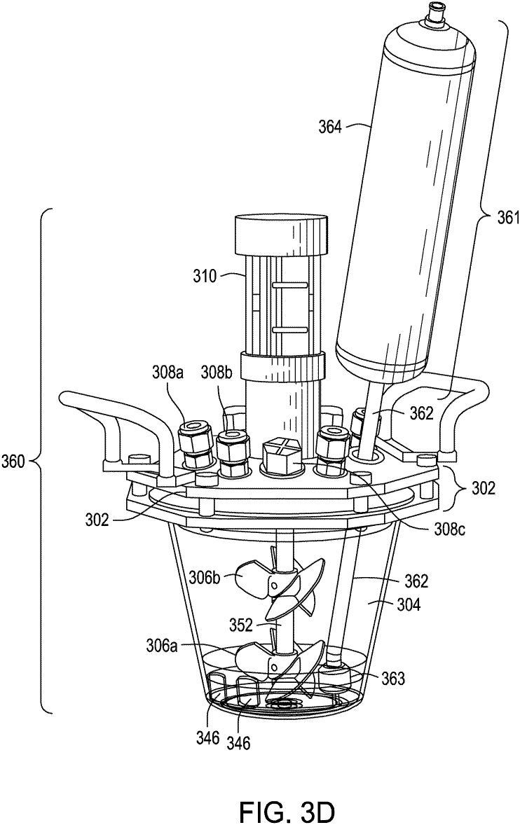

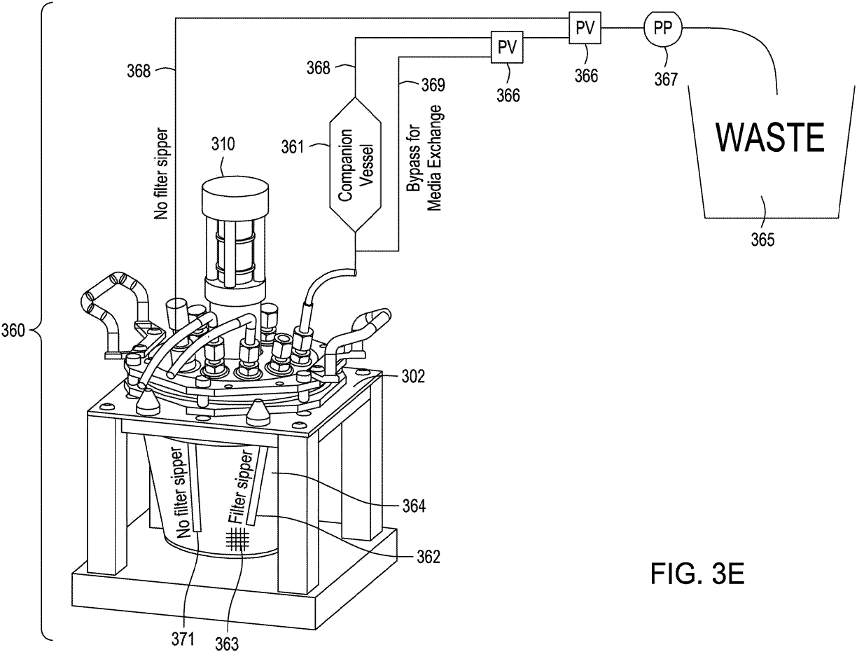

In addition to preparing editing cassettes, cells of choice are made electrocompetent for transformation. The cells that can be edited include any prokaryotic, archaeal or eukaryotic cell. For example, prokaryotic cells for use with the present illustrative embodiments can be gram positive bacterial cells, e.g., Bacillus subtilis, or gram-negative bacterial cells, e.g., E. coli cells. Eukaryotic cells for use with the automated multi-module cell editing instruments of the illustrative embodiments include any plant cells and any animal cells, e.g. fungal cells, insect cells, amphibian cells nematode cells, or mammalian cells. FIGS. 3A to 3E illustrate a bioreactor and other components of the automated system of the disclosure suitable for use with the transcriptional roadblocks disclosed herein.

Once the cells of choice are rendered electrocompetent, the cells and editing cassettes comprising transcriptional roadblocks, such as the ones illustrated in FIG. 1A -FIG. 1C are combined and the editing cassettes are transformed into (e.g., electroporated into) the cells. The cells may be also transformed simultaneously with a separate engine vector expressing an editing nuclease; alternatively and preferably, the cells may already have been transformed with an engine vector configured to express the nuclease; that is, the cells may have already been transformed with an engine vector or the coding sequence for the nuclease may be stably integrated into the cellular genome such that only the editing vector needs to be transformed into the cells.

### Transformation

As used herein, transformation is intended to generically include a variety of art-recognized techniques for introducing an exogenous nucleic acid sequence (e.g., an engine and/or editing vector) into a target cell, and the term “transformation” as used herein includes all transformation and transfection techniques. Such methods include, but are not limited to, electroporation, lipofection, optoporation, injection, microprecipitation, microinjection, liposomes, particle bombardment, sonoporation, laser-induced poration, bead transfection, calcium phosphate or calcium chloride co-precipitation, or DEAE-dextran-mediated transfection. Cells can also be prepared for vector uptake using, e.g., a sucrose, sorbitol or glycerol wash. Additionally, hybrid techniques that exploit the capabilities of mechanical and chemical transfection methods can be used, e.g., magnetofection, a transfection methodology that combines chemical transfection with mechanical methods. In another example, cationic lipids may be deployed in combination with gene guns or electroporators. Suitable materials and methods for transforming or transfecting target cells can be found, e.g., in Green and Sambrook, Molecular Cloning: A Laboratory Manual, 4th ed., Cold Spring Harbor Laboratory Press, Cold Spring Harbor, N.Y. (2014).

Once transformed, the cells are allowed to recover and selection is performed to select for cells transformed with the editing vector, which most often comprises a selectable marker. At a next step , editing is allowed to take place. If one or both components of the editing machinery (e.g., editing cassette and nuclease) is under the control of an inducible promoter, conditions are provided to induce editing. If none of the components of the editing machinery are under the control of an inducible promoter, editing proceeds immediately after transformation. A number of gene regulation control systems have been developed for the controlled expression of (induced by the addition of 2,4 diacetylphloroglucinol (DAPG)), the pBAD promoter (induced by the addition of arabinose to the cell growth medium), and the rhamnose inducible promoter (induced by the addition of rhamnose to the cell growth medium). Other systems include the tetracycline-controlled transcriptional activation system (Tet-On/Tet-Off, Clontech, Inc. (Palo Alto, CA); Bujard and Gossen, PNAS, 89(12):5547-5551 (1992)), the Lac Switch Inducible system (Wyborski et al., Environ Mol Mutagen, 28(4):447-58 (1996); DuCoeur et al., Strategies 5(3):70-72 (1992); U.S. Pat. No. 4,833,080), the ecdysone-inducible gene expression system (No et al., PNAS, 93(8):3346-3351 (1996)), the cumate gene-switch system (Mullick et al., BMC Biotechnology, 6:43 (2006)), and the tamoxifen-inducible gene expression (Zhang et al., Nucleic Acids Research, 24:543-548 (1996)) as well as others. The present compositions and methods preferably make use of rationally-designed editing cassettes such as CREATE TRANSCRIPTIONAL ROADBLOCK cassettes, as described illustrated in FIG. 1A - FIG. 1D and described throughout this specification. FIG. 1D illustrates the initial characterization of complex formation via native PAGE analysis. RNA production is observed in the presence of RNAP. SA binding clearly observed in gel shift of biotinylated DNA. RNA/DNA degradation ws observed in the presence of MAD7 alone and enhanced by the production of gRNA in the reaction. See FIG. 1D. Formation of the roadblock by depicting the gel shift observed when RNAP, Streptavidin, and MAD7 are added to a repair template (i.e., donor HA). RNAP on its own also shifts the mobility of the repair template in the gel shift analysis. Each editing cassette comprises an editing gRNA and a repair DNA comprising an intended edit and a PAM or spacer mutation; thus, e.g., a two-cassette multiplex editing cassette comprises a first editing gRNA and a first editing repair DNA comprising a first intended edit and a first PAM or spacer mutation; and at least a second editing gRNA and at least a second repair DNA comprising at least a second intended edit and a second PAM or spacer mutation. In some embodiments, a single promoter may drive transcription of both the first and second editing gRNAs and both the first and second repair DNAs, and in some embodiments, separate promoters may drive transcription of the first editing gRNA and first repair DNA, and transcription of the second editing gRNA and second repair DNA. In addition, multiplex editing cassettes may comprise nucleic acid elements between the editing cassettes with, e.g., primer sequences, bridging oligonucleotides, and other “cassette-connecting” sequence elements that allow for the assembly of the multiplex editing cassettes.

Once editing is induced, the cells are grown until the cells enter (or are close to entering) the stationary phase of growth, followed by inducing curing of the editing vector by activating an inducible promoter driving transcription of the curing gRNA and inducing the inducible promoter driving transcription of the nuclease. It has been found that curing is particularly effective if the edited cells are in the stationary phase of growth. In yet some aspects, the cells are grown for at least 75% of log phase, 80% of log phase, 85% of log phase, 90% of log phase, 95% of log phase, or are in a stationary phase of growth before inducing curing. Once the editing vector has been cured, the cells are allowed to recover and grow, and then the cells are made electrocompetent once again, ready for another round of editing.

### Automated Cell Editing Instruments and Modules to Perform Nucleic Acid-Guided Nuclease Editing Including Curing

### Automated Cell Editing Instruments

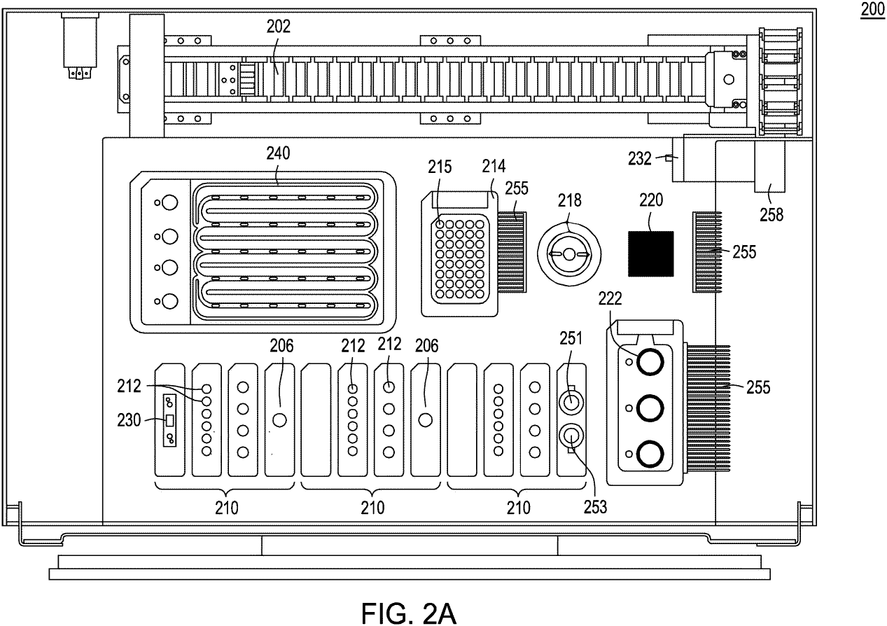

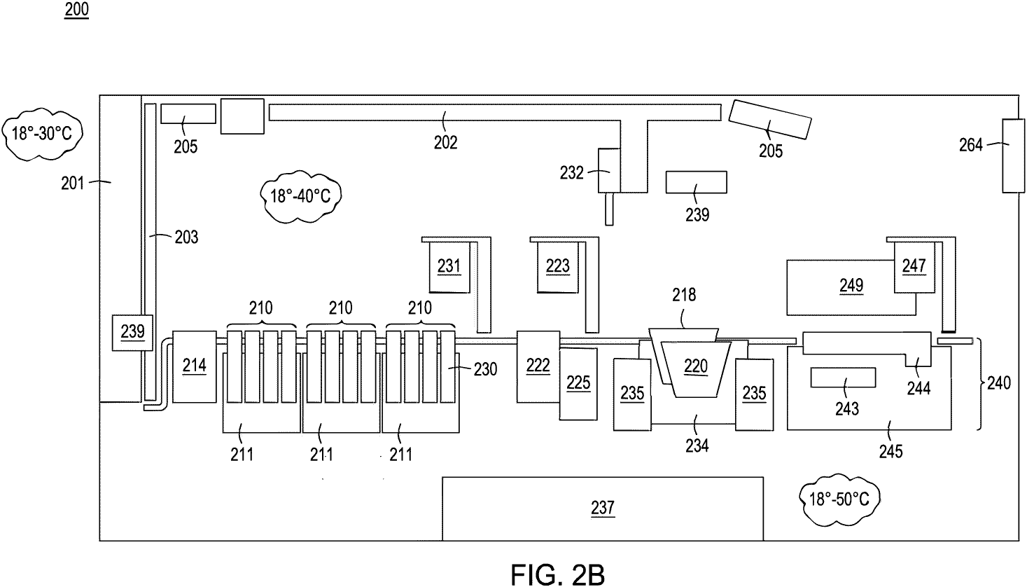

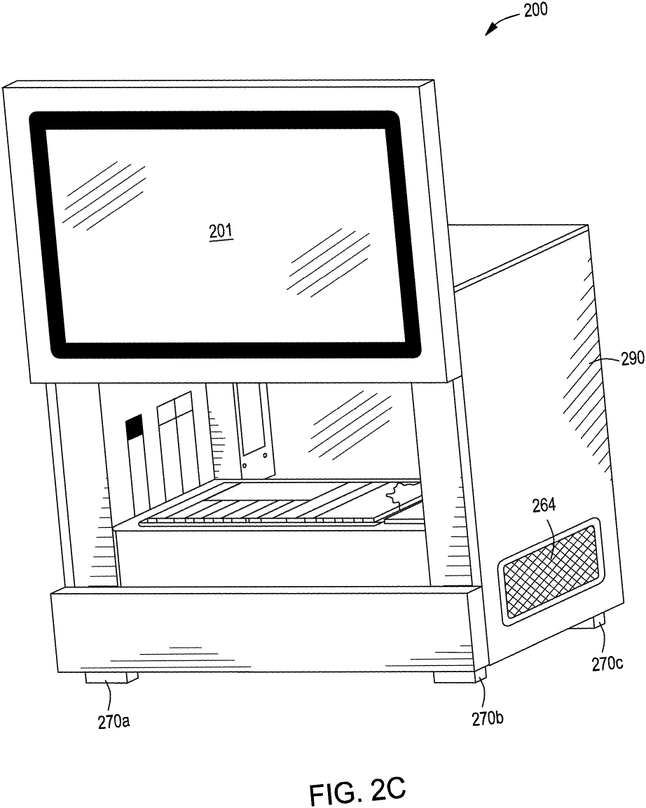

FIG. 2A depicts an exemplary automated multi-module cell processing instrument 200 to, e.g., perform targeted gene editing of live cells. The instrument 200, for example, may be and preferably is designed as a stand-alone benchtop instrument for use within a laboratory environment. The instrument 200 may incorporate a mixture of reusable and disposable components for performing the various integrated processes in conducting automated genome cleavage and/or editing in cells without human intervention. Illustrated is a gantry 202, providing an automated mechanical motion system (actuator) (not shown) that supplies XYZ axis motion control to, e.g., an automated (i.e., robotic) liquid handling system 258 including, e.g., an air displacement pipettor 232 which allows for cell processing among multiple modules without human intervention. In some automated multi-module cell processing instruments, the air displacement pipettor 232 is moved by gantry 202 and the various modules and reagent cartridges remain stationary; however, in other embodiments, the liquid handling system 258 may stay stationary while the various modules and reagent cartridges are moved. Also included in the automated multi-module cell processing instrument 200 are reagent cartridges 210 (see, U.S.P.Ns 10,376,889; 10,406,525; 10,478,822; 10,576,474; 10,639,637; 10,738,271; and 10,799,868) comprising reservoirs 212 and transformation module 230 (e.g., a flow-through electroporation device as described in U.S.P.Ns 10,435,713; 10,443,074; and 10,851,389, as well as wash reservoirs 206, cell input reservoir 251 and cell output reservoir 253. The wash reservoirs 206 may be configured to accommodate large tubes, for example, wash solutions, or solutions that are used often throughout an iterative process. Although two of the reagent cartridges 210 comprise a wash reservoir 206 in FIG. 2A, the wash reservoirs instead could be included in a wash cartridge where the reagent and wash cartridges are separate cartridges. In such a case, the reagent cartridge and wash cartridge may be identical except for the consumables (reagents or other components contained within the various inserts) inserted therein.

In some implementations, the reagent cartridges 210 are disposable kits comprising reagents and cells for use in the automated multi-module cell processing/editing instrument 200. For example, a user may open and position each of the reagent cartridges 210 comprising various desired inserts and reagents within the chassis of the automated multi-module cell editing instrument 200 prior to activating cell processing. Further, each of the reagent cartridges 210 may be inserted into receptacles in the chassis having different temperature zones appropriate for the reagents contained therein.

Also illustrated in FIG. 2A is the robotic liquid handling system 258 including the gantry 202 and air displacement pipettor 232. In some examples, the robotic handling system 258 may include an automated liquid handling system such as those manufactured by Tecan Group Ltd. of Mannedorf, Switzerland, Hamilton Company of Reno, NV (see, e.g., WO2018015544A1), or Beckman Coulter, Inc. of Fort Collins, CO. (see, e.g., US20160018427A1). Pipette tips 215 may be provided in a pipette transfer tip supply 214 for use with the air displacement pipettor 232. The robotic liquid handling system allows for the transfer of liquids between modules without human intervention.

Inserts or components of the reagent cartridges 210, in some implementations, are marked with machine-readable indicia (not shown), such as bar codes, for recognition by the robotic handling system 258. For example, the robotic liquid handling system 258 may scan one or more inserts within each of the reagent cartridges 210 to confirm contents. In other implementations, machine-readable indicia may be marked upon each reagent cartridge 210, and a processing system (not shown, but see element 237 of FIG. 2B) of the automated multi-module cell editing instrument 200 may identify a stored materials map based upon the machine-readable indicia. In the embodiment illustrated in FIG. 2A, a cell growth module comprises a cell growth vial 218 (for details, see U.S.P.Ns 10,435,662; 10,433,031; 10,590,375; 10,717,959; and 10,883,095). Additionally seen is a tangential flow filtration (TFF) module 222 (for details, see U.S.S.Ns 16/516,701 and 16/798,302). Also illustrated as part of the automated multi-module cell processing instrument 200 of FIG. 2A is a singulation module 240 (e.g., a solid wall isolation, incubation and normalization device (SWIIN device)) shown here and described in detail in USPNs 10,533,152; 10,633,626; 10,633,627; 10,647,958; 10,723,995; 10,801,008; 10,851,339; 10,954,485; 10,532,324; 10,625,212; 10,774,462; and 10,835,869), served by, e.g., robotic liquid handing system 258 and air displacement pipettor 232. Additionally seen is a selection module 220 which may employ magnet separation. Also note the placement of three heatsinks 255.

FIG. 2B is a simplified representation of the contents of the exemplary multi-module cell processing instrument 200 depicted in FIG. 2A. Cartridge-based source materials (such as in reagent cartridges 210), for example, may be positioned in designated areas on a deck of the instrument 200 for access by an air displacement pipettor 232.The deck of the multi-module cell processing instrument 200 may include a protection sink (not shown) such that contaminants spilling, dripping, or overflowing from any of the modules of the instrument 200 are contained within a lip of the protection sink. Also seen are reagent cartridges 210, which are shown disposed with thermal assemblies 211 which can create temperature zones appropriate for different reagents in different regions. Note that one of the reagent cartridges also comprises a flow-through electroporation device 230 (FTEP), served by FTEP interface (e.g., manifold arm) and actuator 231. Also seen is TFF module 222 with adjacent thermal assembly 225, where the TFF module is served by TFF interface (e.g., manifold arm) and actuator 223. Thermal assemblies 225, 235, and 245 encompass thermal electric devices such as Peltier devices, as well as heatsinks, fans and coolers. As in FIG. 2A, gantry 202, tip supply 214, cameras 239 and cooling grate 264 are seen.

The rotating growth vial 218 is within a growth module 234, where the growth module is served by two thermal assemblies 235. A selection module is seen at 220. Also seen is the SWIIN module 240, comprising a SWIIN cartridge 244, where the SWIIN module also comprises a thermal assembly 245, illumination 243 (in this embodiment, backlighting), evaporation and condensation control 249, and where the SWIIN module is served by SWIIN interface (e.g., manifold arm) and actuator 247. Also seen in this view is touch screen display 201, display actuator 203, illumination 205 (one on either side of multi-module cell processing instrument 200), and cameras 239 (one camera on either side of multi-module cell processing instrument 200). Finally, element 237 comprises electronics, such as a processor, circuit control boards, high-voltage amplifiers, power supplies, and power entry; as well as pneumatics, such as pumps, valves and sensors.

FIG. 2C illustrates a front perspective view of multi-module cell processing instrument 200 for use in as a benchtop version of the automated multi-module cell editing instrument 200. For example, a chassis 290 may have a width of about 24-48 inches, a height of about 24-48 inches and a depth of about 24-48 inches. Chassis 290 may be and preferably is designed to hold all modules and disposable supplies used in automated cell processing and to perform all processes required without human intervention; that is, chassis 290 is configured to provide an integrated, stand-alone automated multi-module cell processing instrument. As illustrated in FIG. 2C, chassis 290 includes touch screen display 201, cooling grate 264, which allows for air flow via an internal fan (not shown). The touch screen display provides information to a user regarding the processing status of the automated multi-module cell editing instrument 200 and accepts inputs from the user for conducting the cell processing. In this embodiment, the chassis 290 is lifted by adjustable feet 270a, 270b, 270c and 270d (feet 270a-270c are shown in this FIG. 2C). Adjustable feet 270a-270d, for example, allow for additional air flow beneath the chassis 290.

Inside the chassis 290, in some implementations, will be most or all of the components described in relation to FIGS. 2A and 2B, including the robotic liquid handling system disposed along a gantry, reagent cartridges 210 including a flow-through electroporation device, a rotating growth vial 218 in a cell growth module 234, a tangential flow filtration module 222, a SWIIN module 240 as well as interfaces and actuators for the various modules. In addition, chassis 290 houses control circuitry, liquid handling tubes, air pump controls, valves, sensors, thermal assemblies (e.g., heating and cooling units) and other control mechanisms. For examples of multi-module cell editing instruments, see U.S.P.Ns 10,253,316; 10,329,559; 10,323,242; 10,421,959; 10,465,185; 10,519,437; 10,584,333; 10,584,334; 10,647,982; 10,689,645; 10,738,301; 10,738,663; 10,947,532; 10,894,958; 10,954,512; and 11,034,953, all of which are herein incorporated by reference in their entirety.

### Alternative Embodiment of an Automated Cell Editing Instrument

A bioreactor may be used to grow cells-in particular mammalian cells-off-instrument or to allow for cell growth and recovery on-instrument; e.g., as one module of a multi-module fully-automated closed instrument. Further, the bioreactor supports cell selection/enrichment, via expressed antibiotic markers in the growth process or via expressed antibodies coupled to magnetic beads and a magnet associated with the bioreactor. There are many bioreactors known in the art, including those described in, e.g., WO 2019/046766; 10,699,519; 10,633,625; 10,577,576; 10,294,447; 10,240,117; 10,179,898; 10,370,629; and 9,175,259; and those available from Lonza Group Ltd. (Basel, Switzerland); Miltenyi Biotec (Bergisch Gladbach, Germany), Terumo BCT (Lakewood, CO) and Sartorius GmbH (Gottingen, Germany).

FIG. 3A shows one embodiment of a bioreactor assembly 300 suitable for cell growth, transfection, and editing as one component of an automated multi-module cell processing instrument. Unlike most bioreactors that are used to support fermentation or other processes with an eye to harvesting the products produced by organisms grown in the bioreactor, the present bioreactor (and the processes performed therein) is configured to grow cells, monitor cell growth (via, e.g., optical means or capacitance), passage cells, select cells, transfect cells, and support the growth and harvesting of edited cells. Bioreactor assembly 300 comprises cell growth vessel 301 comprising a main body 304 with a lid assembly 302 comprising ports 308, including a motor integration port 310 configured to accommodate a motor to drive impeller 306 via impeller shaft 352 and propellers 306a and 306b. The tapered shape of main body 304 of the growth vessel 301 along with, in some embodiments, dual impellers allows for working with a larger dynamic range of volumes, such as, e.g., up to 500 ml and as low as 100 ml for rapid sedimentation of the microcarriers.

Bioreactor assembly 300 further comprises bioreactor stand assembly 303 comprising a main body 312 and growth vessel holder 314 comprising a heat jacket or other heating means (not shown) into which the main body 304 of growth vessel 301 is disposed in operation. The main body 304 of growth vessel 301 is biocompatible and preferably transparent-in some embodiments, in the UV and IR range as well as the visible spectrum-so that the growing cells can be visualized by, e.g., cameras or sensors integrated into lid assembly 302 or through viewing apertures or slots 346 in the main body 312 of bioreactor stand assembly 303. Camera mounts are shown at 344.

Bioreactor assembly 300 supports growth of cells from a 500,000 cell input to a 10 billion cell output, or from a 1 million cell input to a 25 billion cell output, or from a 5 million cell input to a 50 billion cell output or combinations of these ranges depending on, e.g., the size of main body 304 of growth vessel 301, the medium used to grow the cells, the type and size and number of microcarriers used for growth (if microcarriers are used), and whether the cells are adherent or non-adherent. The bioreactor that comprises assembly 300 supports growth of both adherent and non-adherent cells, wherein adherent cells are typically grown of microcarriers as described in detail in U.S.S.N 17/237,747, filed 24 Apr. 2021. Alternatively, another option for growing mammalian cells in the bioreactor described herein is growing single cells in suspension using a specialized medium such as that developed by ACCELLTA™ (Haifa, Israel). Cells grown in this medium must be adapted to this process over many cell passages; however, once adapted the cells can be grown to a density of >40 million cells/ml and expanded 50-100x in approximately a week, depending on cell type.

Main body 304 of growth vessel 301 preferably is manufactured by injection molding, as is, in some embodiments, impeller 306 and the impeller shaft 352. Impeller 306 also may be fabricated from stainless steel, metal, plastics or the polymers listed infra. Injection molding allows for flexibility in size and configuration and also allows for, e.g., volume markings to be added to the main body 304 of growth vessel 301. Additionally, material from which the main body 304 of growth vessel 301 is fabricated should be able to be cooled to about 4° C. or lower and heated to about 55° C. or higher to accommodate cell growth. Further, the material that is used to fabricate the vial preferably is able to withstand temperatures up to 55° C. without deformation. Suitable materials for main body 304 of growth vessel 301 include cyclic olefin copolymer (COC), glass, polyvinyl chloride, polyethylene, polyetheretherketone (PEEK), polypropylene, polycarbonate, poly(methyl methacrylate (PMMA)), polysulfone, poly(dimethylsiloxane), cyclo-olefin polymer (COP), and co-polymers of these and other polymers. Preferred materials include polypropylene, polycarbonate, or polystyrene. The material used for fabrication may depend on the cell type to be grown, transfected and edited, and be conducive to growth of both adherent and non-adherent cells and workflows involving microcarrier-based transfection. The main body 304 of growth vessel 301 may be reusable or, alternatively, may be manufactured and configured for a single use. In one embodiment, main body 304 of growth vessel 301 may support cell culture volumes of 25 ml to 500 ml, but may be scaled up to support cell culture volumes of up to 3 L.

The bioreactor stand assembly comprises a stand or frame 350 and a main body 312 that holds the growth vessel 301 during operation. The stand/frame 350 and main body 312 are fabricated from stainless steel, other metals, or polymer/plastics. The bioreactor stand assembly main body further comprises a heat jacket (not seen in FIG. 3A) to maintain the growth vessel main body 304—and thus the cell culture-at a desired temperature. Additionally, the stand assembly can host a set of sensors and cameras (camera mounts are shown at 344) to monitor cell culture.

FIG. 3B depicts a top-down view of one embodiment of vessel lid assembly 302. Growth vessel lid assembly 302 is configured to be air-tight, providing a sealed, sterile environment for cell growth, transfection and editing as well as to provide biosafety in a closed system. Vessel lid assembly 302 and the main body of growth vessel can be reversibly sealed via fasteners such as screws, or permanently sealed using biocompatible glues or ultrasonic welding. Vessel lid assembly 302 in some embodiments is fabricated from stainless steel such as S316L stainless steel but may also be fabricated from metals, other polymers (such as those listed supra) or plastics. As seen in this FIG. 3B—as well as in FIG. 3A—vessel lid assembly 302 comprises a number of different ports to accommodate liquid addition and removal; gas addition and removal; for insertion of sensors to monitor culture parameters (described in more detail infra); to accommodate one or more cameras or other optical sensors; to provide access to the main body 304 of growth vessel 301 by, e.g., a liquid handling device; and to accommodate a motor for motor integration to drive one or more impellers 306. Exemplary ports depicted in FIG. 3B include three liquid-in ports 316 (at 4 o′clock, 6 o′clock and 8 o′clock); two self-sealing ports 317, 330 (at 3 o′clock and at 7 o′clock) to provide access to the main body 304 of growth vessel 301; one liquid-out port 322 (at 11 o′clock); a capacitance sensor 318 (at 9 o′clock); one “gas in” port 324 (at 12 o′clock); one “gas out” port 320 (at 10 o′clock); an optical sensor 326 (at 1 o′clock); a rupture disc 328 at 2 o′clock; and (a temperature probe 332 (at 5 o′clock).

The ports shown in vessel lid assembly 302 in this FIG. 3B are exemplary only and it should be apparent to one of ordinary skill in the art given the present disclosure that, e.g., a single liquid-in port 316 could be used to accommodate addition of all liquids to the cell culture rather than having a liquid-in port for each different liquid added to the cell culture. Further, any liquid-in port may serve as both a liquid-in port and a liquid-out port. Similarly, there may be more than one gas-in port 324, such as one for each gas, e.g., O₂, CO₂ that may be added. In addition, although a temperature probe 332 is shown, a temperature probe alternatively may be located on the outside of vessel holder 314 of bioreactor stand assembly 303 separate from or integrated into heater jacket (not seen in this FIG. 3B). A self-sealing port 330, if present, allows access to the main body 304 of growth vessel 301 for, e.g., a pipette, syringe, or other liquid delivery system via a gantry (not shown). As shown in FIG. 3A, additionally there may be a motor integration port 310 to drive the impeller(s), although other configurations of growth vessel 301 may alternatively integrate the motor drive at the bottom of the main body 304 of growth vessel 301. Growth vessel lid assembly 302 may also comprise a camera port for viewing and monitoring the cells.

Additional sensors include those that detect dissolved O₂ concentration, dissolved CO₂ concentration, culture pH, lactate concentration, glucose concentration, biomass, and optical density. The sensors may use optical (e.g., fluorescence detection), electrochemical, or capacitance sensing and either be reusable or configured and fabricated for single-use. Sensors appropriate for use in the bioreactor are available from Omega Engineering (Norwalk CT); PreSens Precision Sensing (Regensburg, Germany); C-CIT Sensors AG (Waedenswil, Switzerland), and ABER Instruments Ltd. (Alexandria, VA). In one embodiment, optical density is measured using a reflective optical density sensor to facilitate sterilization, improve dynamic range and simplify mechanical assembly.

The rupture disc, if present, provides safety in a pressurized environment, and is programmed to rupture if a threshold pressure is exceeded in growth vessel. If the cell culture in the growth vessel is a culture of adherent cells, microcarriers may be used as described in U.S.S.N 17/237,747, filed 24 Apr. 2021. In such an instance, the liquid-out port may comprise a filter such as a stainless steel or plastic (e.g., polyvinylidene difluoride (PVDF), nylon, polypropylene, polybutylene, acetal, polyethylene, or polyamide) filter or frit to prevent microcarriers from being drawn out of the culture during, e.g., medium exchange, but to allow dead cells to be withdrawn from the vessel. Additionally, a liquid port may comprise a filter sipper to allow cells that have been dissociated from microcarriers to be drawn into the cell corral while leaving spent microcarriers in main body 304 of growth vessel 301. The microcarriers used for initial cell growth can be nanoporous (where pore sizes are typically <20 nm in size), microporous (with pores between >20 nm to <1 µm in size), or macroporous (with pores between >1 µm in size, e.g. 20 µm) and the microcarriers are typically 50-200 µm in diameter; thus the pore size of the filter or frit in the liquid-out port will differ depending on microcarrier size.

The microcarriers used for cell growth depend on cell type and desired cell numbers, and typically include a coating of a natural or synthetic extracellular matrix or cell adhesion promoters (e.g., antibodies to cell surface proteins or poly-L-lysine) to promote cell growth and adherence. Microcarriers for cell culture are widely commercially available from, e.g., Millipore Sigma, (St. Louis, MO, USA); ThermoFisher Scientific (Waltham, MA, USA); Pall Corp. (Port Washington, NY, USA); GE Life Sciences (Marlborough, MA, USA); and Corning Life Sciences (Tewkesbury, MA, USA). As for the extracellular matrix, natural matrices include collagen, fibrin and vitronectin (available, e.g., from ESBio, Alameda, CA, USA), and synthetic matrices include MATRIGEL® (Corning Life Sciences, Tewkesbury, MA, USA), GELTREX™ (ThermoFisher Scientific, Waltham, MA, USA), CULTREX® (Trevigen, Gaithersburg, MD, USA), biomemetic hydrogels available from Cellendes (Tubingen, Germany); and tissue-specific extracellular matrices available from Xylyx (Brooklyn, NY, USA); further, denovoMatrix (Dresden, Germany) offers screenMATRIX™, a tool that facilitates rapid testing of a large variety of cell microenvironments (e.g., extracellular matrices) for optimizing growth of the cells of interest.

FIG. 3C is a side perspective view of the assembled bioreactor 342 without sensors mounted in ports 308. Seen are vessel lid assembly 302, bioreactor stand assembly 303, bioreactor stand main body 312 into which the main body of growth vessel 301 (not seen in this FIG. 3C) is inserted. Also present are two camera mounts 344, a motor integration port 310, and stand or frame 350.

FIG. 3D shows the embodiment of a bioreactor/cell corral assembly 360, comprising the bioreactor assembly 300 for cell growth, transfection, and editing described in FIG. 3A and further comprising a cell corral 361. Bioreactor assembly 300 comprises a growth vessel 301 (not labeled in this FIG. D) comprising tapered a main body 304 with a lid assembly 302 comprising ports 308 (here, 308a, 308b, 308c), including a motor integration port 310 driving impeller 306 via impeller shaft 352, as well as two viewing ports 346. Cell corral 361 comprises a main body 364, and end caps, where the end cap proximal the bioreactor assembly 300 is coupled to a filter sipper 362 comprising a filter portion 363 disposed within the main body 304 of the bioreactor assembly 300. The filter sipper is disposed within the main body 304 of the bioreactor assembly 300 but does not reach to the bottom surface of the bioreactor assembly 300 to leave a “dead volume” for spent microcarriers to settle while cells are removed from the growth vessel 301 into the cell corral 361. The cell corral may or may not comprise a temperature or CO₂ probe, and may or not be enclosed within an insulated jacket.

The cell corral 361, like the main body 304 of growth vessel 301 is fabricated from any biocompatible material such as polycarbonate, cyclic olefin copolymer (COC), glass, polyvinyl chloride, polyethylene, polyetheretherketone (PEEK), polypropylene, poly(methyl methacrylate (PMMA)), polysulfone, poly(dimethylsiloxane), cyclo-olefin polymer (COP), and co-polymers of these and other polymers. Likewise, the end caps of the cell corral are fabricated from a biocompatible material such as polycarbonate, cyclic olefin copolymer (COC), glass, polyvinyl chloride, polyethylene, polyetheretherketone (PEEK), polypropylene, poly(methyl methacrylate (PMMA)), polysulfone, poly(dimethylsiloxane), cyclo-olefin polymer (COP), and co-polymers of these and other polymers. The cell corral may be coupled to or integrated with one or more devices, such as a flow cell where an aliquot of the cell culture can be counted. Additionally, the cell corral may comprise additional liquid ports for adding medium, other reagents, and/or fresh microcarriers to the cells in the cell corral. The volume of the main body 364 of the cell corral 361 may be from 25 to 3000 mL, or from 250 to 1000 mL, or from 450 to 500 mL.

In operation, the bioreactor/cell corral assembly 360 comprising the bioreactor assembly 300 and cell corral 361 grows, passages, transfects, and supports editing and further growth of mammalian cells (note, the bioreactor stand assembly is not shown in this FIG. 3D). Cells are transferred to the growth vessel 301 comprising medium and microcarriers. The cells are allowed to adhere to the microcarries. Approximately 2000,000 microcarriers (e.g., laminin-521 coated polystyrene with enhanced attachment surface treatment) are used for the initial culture of approximately 20 million cells to where there are approximately 50 cells per microcarrier. The cells are grown until there are approximately 500 cells per microcarrier. For medium exchange, the microcarriers comprising the cells are allowed to settle and spent medium is aspirated via a sipper filter, wherein the filter has a mesh small enough to exclude the microcarriers. The mesh size of the filter will depend on the size of the microcarriers and cells present but typically is from 50 to 500 µm, or from 70 to 200 µm, or from 80 to 110 µm. For passaging the cells, the microcarriers are allowed to settle and spent medium is removed from the growth vessel 301, and phosphobuffered saline or another wash agent is added to the growth vessel 301 to wash the cells on the microcarriers. Optionally, the microcarriers are allowed to settle once again, and some of the wash agent is removed. At this point, the cells are dissociated from the microcarriers. Dissociation may be accomplished by, e.g., bubbling gas or air through the wash agent in the growth vessel 301, by increasing the impeller speed and/or direction, by enzymatic action (via, e.g., trypsin), or by a combination of these methods. In one embodiment, a chemical agent such as the RelesR™ reagent (STEMCELL Technologies Canada INC., Vancouver, BC) is added to the microcarriers in the remaining wash agent for a period of time required to dissociate most of the cells from the microcarriers, such as from 1 to 60 minutes, or from 3 to 25 minutes, or from 5 to 10 minutes. Once enough time has passed to dissociate the cells, cell growth medium is added to the growth vessel 301 to stop the enzymatic reaction.

Once again, the now-spent microcarriers are allowed to settle to the bottom of the growth vessel 301 and the cells are aspirated through a filter sipper into the cell corral 361. The growth vessel 301 is configured to allow for a “dead volume” of 2 mL to 200 mL, or 6 mL to 50 mL, or 8 mL to 12 mL below which the filter sipper does not aspirate medium to ensure the settled spent microcarriers are not transported to the filter sipper during fluid exchanges. Once the cells are aspirated from the bioreactor vessel leaving the “dead volume” of medium and spent microcarriers, the spent microcarriers are aspirated through a non-filter sipper into waste. The spent microcarriers (and the bioreactor vessel) are diluted in phosphobuffered saline or other buffer one or more times, wherein the wash agent and spent microcarriers continue to be aspirated via the non-filter sipper leaving a clean bioreactor vessel. After washing, fresh microcarriers or RBMCs and fresh medium are dispensed into the bioreactor vessel and the cells in the cell corral are dispensed back into the bioreactor vessel for another round of passaging or for transfection and editing, respectively.

FIG. 3E depicts a bioreactor and bioreactor/cell corral assembly 360 comprising a growth vessel 301, with a main body 364, lid assembly 302 comprising a motor integration port 310, a filter sipper 362 comprising a filter 363 and a non-filter sipper 371, 368. Also seen is a cell corral 361, fluid line 368 from the cell corral through pinch valve 366, and a line 369 for medium exchange also connected to a pinch valve 366. The non-filter sipper 368 also runs through a pinch valve 366 to waste 365. Also seen is a peristaltic pump 367.

### EXAMPLES

The following examples are put forth so as to provide those of ordinary skill in the art with a complete disclosure and description of how to make and use the present invention, and are not intended to limit the scope of what the inventors regard as their invention, nor are they intended to represent or imply that the experiments below are all of or the only experiments performed. It will be appreciated by persons skilled in the art that numerous variations and/or modifications may be made to the invention as shown in the specific aspects without departing from the spirit or scope of the invention as broadly described. The present aspects are, therefore, to be considered in all respects as illustrative and not restrictive.

### Example I: Establishing In-Vitro Conditions for Generating RNAP Roadblocks as a Mechanism for Tethering Repair DNA to Guide/RNP Complex

Transcriptional roadblocks with CREATE/RNAP-tethered bundles were generated from a pCOMPLETE2 backbone and J23119-G2B insert with or without a biotinylated 3′ end. Briefly, streptavidin (SA), MAD7, and E. coli RNAP were added to the pCOMPLETE2 backbones with or without the biotinylated 3′ ends under standard conditions for supporting transcription by the RNAP.

The reaction set up was as follows:

TABLE 1

| Stock Reagents | | Reaction Concentration | |
|---|---|---|---|
| RNAP | 1 | 0.05 | U/uL |
| MAD7 | 5 | 250 | nM |
| Streptavidin | 20 | 1000 | nM |
| Insert | 50 | 30.3030303 | nM |
| Bb | 100 | 3.03030303 | nM |
| NTP | 5 | 0.25 | mM |

The reactions were run was outlined below:

TABLE 2

| Order of Operations | 1 | 2 | 3 | 4 | 5 | 6 | 7 | |
|---|---|---|---|---|---|---|---|---|
| Insert Description | ddH₂O | 5X Txn buffer | Template DNA uL | NTP vol uL | MAD7 vol uL | Strep vol uL | RNAP vol uL | Total vol |
| 1G2B_5Acrd/3 PT | 16.00 | 2 | 1 | 1 | 0 | 0 | 0 | 20.00 |
| A2G2B_5Acrd/ 3PT | 15.00 | 2 | 1 | 1 | 0 | 1 | 0 | 20.00 |
| A3G2B_5Acrd/ 3PT | 15.00 | 2 | 1 | 1 | 1 | 0 | 0 | 20.00 |
| A4G2B_5Acrd/ 3PT | 15.00 | 2 | 1 | 1 | 0 | 0 | 1 | 20.00 |
| A5G2B_5Acrd/ 3PT | 14.00 | 2 | 1 | 1 | 0 | 1 | 1 | 20.00 |
| A6G2B_5Acrd/ 3PT | 13.00 | 2 | 1 | 1 | 1 | 1 | 1 | 20.00 |
| A7G2B_5Acrd/ 3BiotinPT | 16.00 | 2 | 1 | 1 | 0 | 0 | 0 | 20.00 |
| A8G2B_5Acrd/ 3BiotinPT | 15.00 | 2 | 1 | 1 | 0 | 1 | 0 | 20.00 |
| A9G2B_5Acrd/ 3BiotinPT | 15.00 | 2 | 1 | 1 | 1 | 0 | 0 | 20.00 |
| G2B_5Acrd/3Bi otinPT | 15.00 | 2 | 1 | 1 | 0 | 0 | 1 | 20.00 |
| AllG2B_5Acrd /3BiotinPT | 14.00 | 2 | 1 | 1 | 0 | 1 | 1 | 20.00 |
| A12G2B_5Acrd /3BiotinPT | 13.00 | 2 | 1 | 1 | 1 | 1 | 1 | 20.00 |

The reaction was designed to illustrate the generation of a transcriptional roadblock such as the one illustrated in FIG. 1A or FIG. 1B where a dsDNA repair nucleic acid is tethered by an RNA polymerase (RNAP) molecule stalled at a transcriptional roadblock located at a pre-determined location – in this example the biotin-streptavidin roadblock – via binding of the nuclease to RNA transcribed from the dsDNA repair nucleic acid. FIG. 1D illustrates a characterization of the RNAP roadblock complex formation via PAGE analysis. FIG. 1D illustrates RNA production in presence of RNAP. FIG. 1D also shows that a binding reaction is observed in the biotinylated DNA. RNA/DNA degradation is observed in the presence of MAD7 alone and enhanced by the production of gRNA in the reaction, suggesting that the complex may lack stability in vitro.

### Example II: Fully-Automated Singleplex RGN-Directed Editing Run

Singleplex automated genomic editing using MAD7 nuclease was successfully performed with an automated multi-module instrument of the disclosure. For examples of multi-module cell editing instruments, see U.S.P.Ns 10,253,316, issued 09 Apr. 2019; 10,329,559, issued 25 Jun. 2019; 10,323,242, issued 18 Jun. 2019; 10,421,959, issued 24 Sep. 2019; 10,465,185, issued 05 Nov. 2019; 10,519,437, issued 31 Dec. 2019; 10,584,333, issued 10 Mar. 2020; 10,584,334, issued 10 Mar. 2020; 10,647,982, issued 12 May 2020; 10,689,645, issued 23 Jun. 2020; 10,738,301, issued 11 Aug. 2020; and USSNs 16/920,853, filed 06 Jul. 2020; and 16/988,694, filed 09 Aug. 2020, all of which are herein incorporated by reference in their entirety.

An ampR plasmid backbone and a lacZ_F172* editing cassette were assembled via Gibson Assembly® into an “editing vector” in an isothermal nucleic acid assembly module included in the automated instrument. lacZ_F172 functionally knocks out the lacZ gene. “lacZ_F172*” indicates that the edit happens at the 172nd residue in the lacZ amino acid sequence. Following assembly, the product was de-salted in the isothermal nucleic acid assembly module using AMPure® beads, washed with 80% ethanol, and eluted in buffer. The assembled editing vector and recombineering-ready, electrocompetent E. Coli cells are transferred into a transformation module for electroporation. The cells and nucleic acids are combined and allowed to mix for 1 minute, and electroporation was performed for 30 seconds. The parameters for the poring pulse are: voltage, 2400 V; length, 5 ms; interval, 50 ms; number of pulses, 1; polarity, +. The parameters for the transfer pulses are: Voltage, 150 V; length, 50 ms; interval, 50 ms; number of pulses, 20; polarity, +/-. Following electroporation, the cells were transferred to a recovery module (another growth module), and allowed to recover in SOC medium containing chloramphenicol. Carbenicillin was added to the medium after 1 hour, and the cells were allowed to recover for another 2 hours. After recovery, the cells were held at 4° C. until recovered by the user.

After the automated process and recovery, an aliquot of cells is plated on MacConkey agar base supplemented with lactose (as the sugar substrate), chloramphenicol and carbenicillin and grown until colonies appeared. White colonies represented functionally edited cells, purple colonies represented un-edited cells. All liquid transfers are performed by the automated liquid handling device of the automated multi-module cell processing instrument.

While this invention is satisfied by embodiments in many different forms, as described in detail in connection with preferred embodiments of the invention, it is understood that the present disclosure is to be considered as exemplary of the principles of the invention and is not intended to limit the invention to the specific embodiments illustrated and described herein. Numerous variations may be made by persons skilled in the art without departure from the spirit of the invention. The scope of the invention will be measured by the appended claims and their equivalents. The abstract and the title are not to be construed as limiting the scope of the present invention, as their purpose is to enable the appropriate authorities, as well as the general public, to quickly determine the general nature of the invention. In the claims that follow, unless the term “means” is used, none of the features or elements recited therein should be construed as means-plus-function limitations pursuant to 35 U.S.C. §112, ¶6.

1-30. (canceled)

31. A composition for homologous recombination-based editing of a cell comprising:
a double-stranded repair deoxyribonucleic acid (dsDNA) cassette having:
i) a promoter operably linked to (a) a sequence encoding a guide RNA for recruiting an RNA guided endonuclease selected from the group consisting of Cas9 and Cpf1, and (b) a sequence homologous to a target region of a target cell, wherein the sequence homologous to the target region has at least one nucleic acid base variation compared to the target region of the target cell; and
ii) a transcriptional roadblock moiety, wherein the transcriptional roadblock moiety is at a 3′ end of the dsDNA cassette, or within the sequence homologous to the target region of the target cell, wherein the dsDNA cassette is capable of being tethered by an RNA polymerase (RNAP) stalled at the transcriptional roadblock moiety to a transcript comprising the guide RNA.

32. The composition of claim 31, wherein the transcriptional roadblock moiety comprises a non-covalent interaction between a transcriptional roadblock ligand and a transcriptional roadblock ligand-binding moiety.

33. The composition of claim 32, wherein the non-covalent interaction between the transcriptional roadblock ligand and the transcriptional roadblock ligand binding moiety has a dissociation constant (K_{d}) on the order of 10⁻¹² mol/L, on the order of 10⁻¹³ mol/L, or on the order of 10⁻¹⁴ mol/L.

34. The composition of claim 32, wherein the transcriptional roadblock ligand is a biotin molecule.

35. The composition of claim 32, wherein the transcriptional roadblock ligand-binding moiety is a streptavidin molecule or an avidin molecule.

36. The composition of claim 31, wherein the transcriptional roadblock moiety is a non-canonical nucleobase or a stretch of non-canonical nucleobases.

37. The composition of claim 31, wherein the sequence homologous to the target region has between one to five variations, between one to ten variations, between one to fifteen variations, between one to twenty variations, between one to twenty-five variations, between one to thirty variations, between one to thirty-five variations, between one to forty variations, between one to forty-five variations, between one to fifty variations, between one to fifty-five variations, or between one to sixty variations compared to the target cell.

38. The composition of claim 31, wherein the composition comprises a plurality of dsDNA cassettes for multiplex gene editing.

39. The composition of claim 38, wherein the plurality of dsDNA cassettes in the composition are homologous to at least 2, at least 10, at least 50, or at least 100 distinct target regions of the target cell.

40. The composition of claim 38, wherein the plurality of dsDNA cassettes in the composition target an order of 10³ to 10⁵ distinct target regions of the target cell.

41. The composition of claim 31, wherein the variation is a deletion of a nucleobase, an addition of a nucleobase, or a replacement of a nucleobase compared to the target region of the target cell.

42. The composition of claim 31, wherein the at least one nucleic acid base variation compared to the target region of the target cell is designed to introduce a silent mutation in the target cell.

43. The composition of claim 31, wherein the dsDNA cassette further comprises a sequence encoding the RNA guided endonuclease.

44. The composition of claim 42, wherein the silent mutation provides a site conferring immunity to further editing by the RNA guided endonuclease.

45. The composition of claim 44, wherein the site conferring immunity comprises a change in a protospacer adjacent motif (PAM) sequence for the RNA guided endonuclease.

46. The composition of claim 31, wherein the sequence homologous to the target region is between 50 base pairs to 500 base pairs long.

47. The composition of claim 31, wherein the target cell is a human cell.

48. The composition of claim 31, wherein the target cell is a mammalian cell, a bacterial cell, or a yeast cell.

49. The composition of claim 31, wherein the dsDNA cassette is capable of being tethered to the guide RNA by the RNAP with a 1:1 stoichiometry.
